(19) United States
(12) Patent Application Publication
(10) Pub. No.: US 2025/0361511 A1
(43) Pub. Date: Nov. 27, 2025

(54) NANOPARTICLE COMPOSITIONS CONTAINING SIRP-alpha siRNA FOR TREATMENT OF CANCER

(71) Applicants: The Texas A&M University System, College Station, TX (US); The Methodist Hospital System, Houston, TX (US)

(72) Inventors: Shreya Raghavan, College Station, TX (US); Sabrina VandenHeuvel, College Station, TX (US); Eric Chau, Houston, TX (US); Biana Godin, Houston, TX (US)

(21) Appl. No.: 19/219,989

(22) Filed: May 27, 2025

Related U.S. Application Data
(60) Provisional application No. 63/651,765, filed on May 24, 2024.

(51) Int. Cl. C12N 15/113; A61K 9/1272; A61K 9/51; A61K 31/44; A61K 31/555; A61K 33/243; A61P 35/04; C12N 15/88

(52) U.S. Cl. CPC C12N 15/1138; A61K 9/1272; A61K 9/5123; A61K 31/44; A61K 31/555; A61K 33/243; A61P 35/04; C12N 15/88; C12N 2310/11; C12N 2310/14; C12N 2320/31; C12N 2320/32

(57) ABSTRACT

Provided here are nanoparticle compositions containing siRNA to disrupt the signal regulatory protein-α (SIRPα) signaling pathway. Embodiments include methods for treating a subject diagnosed as having ovarian cancer by administering to the subject the nanoparticle composition containing SIRPα siRNA. Other methods include administering to the subject the nanoparticle composition containing SIRPα siRNA in addition to a platinum-based chemotherapeutic agent.

### TECHNICAL FIELD

The disclosure relates to nanoparticle compositions containing short or small interfering RNAs targeting signal regulatory protein-α (SIRPα siRNA).

### BACKGROUND

Ovarian cancer (OvCa) is the most lethal gynecologic malignancy, with high grade serous OvCa as the most common subtype. This high mortality rate is often the result of late diagnosis when OvCa has already spread into the peritoneal cavity and into the liver. There are no established early detection or screening criteria for OvCa. Therefore, diagnosis is typically late when OvCa has already spread significantly into the peritoneal cavity and infiltrated the liver. Up to 50% of women with advanced OvCa have OvCa liver metastases (OCLM). Cytoreductive surgery is often limited by the degree and extent of liver infiltration. Gold-standard platinum chemotherapy rapidly becomes obsolete in metastatic OvCa and OCLM due to the abnormal immune response driving the development of resistance mechanisms, leaving no further options to treat OCLM. Therefore, there is an exigent need to develop new ways to treat advanced OvCa, especially those aimed at eliminating ovarian metastases infiltrating the liver.

### SUMMARY

Provided here are nanoparticle compositions containing siRNA to disrupt the signal regulatory protein-α (SIRPα) signaling pathway. In certain embodiments, these compositions disrupt the signaling pathway between ovarian cancer cells and macrophages to treat advanced ovarian cancer and ovarian cancer metastasis. In certain embodiments, the nanoparticle composition is a lipid nanoparticle. In certain embodiments, the nanoparticle composition is a liposome. Embodiments include a pharmaceutical composition containing a therapeutically effective amount of the nanoparticle composition containing SIRPα siRNA. These siRNAs are used to interfere with gene expression at the post-transcriptional level by cleaving mRNA molecules with complementary sequences.

Embodiments of the SIRPα siRNA include sequences presented as sense and antisense sequences that target one or more portions of the signal regulatory protein-α gene. Embodiments of the SIRPα siRNA include sequences presented as sense and antisense sequence of SASI_Hs01_00145338 that target the gene region starting at position 1458, provided below as SEQ ID NO. 1 and SEQ ID NO. 2.

| | Sense sequence (5′-3′): |
|---|---|
| SEQ ID NO. 1 | |
| | CUAAUGAACGGAACAUCUA[dT][dT] |
| | Antisense sequence (5′-3′): |
| SEQ ID NO. 2 | |
| | UAGAUGUUCCGUUCAUUAG[dT][dT] |

Embodiments of the SIRPα siRNA include sequences presented as sense and antisense sequence of SASI_Hs01_00017994 that target the gene region starting at position 1366, provided below as SEQ ID NO. 3 and SEQ ID NO. 4.

| | Sense sequence (5′-3′): |
|---|---|
| SEQ ID NO. 3 | |
| | CUAAUGAACGGAACAUCUA[dT][dT] |
| | Antisense sequence (5′-3′): |
| SEQ ID NO. 4 | |
| | UAGAUGUUCCGUUCAUUAG[dT][dT] |

Embodiments include methods for treating a metastatic tissue by contacting the tissue with the nanoparticle composition containing SIRPα siRNA. Embodiments include methods for treating a metastatic tissue by contacting the tissue with the nanoparticle composition containing sense or antisense sequences of SASI_Hs01_00145338. Embodiments include methods for treating a metastatic tissue by contacting the tissue with the nanoparticle composition containing sense or antisense sequences of SASI_Hs01_00017994. In certain embodiments, the metastatic tissue is an ovarian cancer tissue.

Metastatic OvCa cells aggregate within the ascites fluid and form spheroids before seeding secondary organs throughout the peritoneal cavity. Provided herein are methods that involve the hanging drop array, which closely mimics the presence of OvCa as non-adherent clusters within the ascites. Within the peritoneal ascites fluid, OvCa cells interact with immune and stromal cells (including macrophages among several other cell types) that significantly impact malignant disease progression. Macrophages are the most abundant innate immune cell population in advanced OvCa. Macrophages drive OvCa invasion and chemoresistance via a variety of reciprocal signaling mechanisms like Wnt, growth factor and chemokine secretions and metabolic crosstalk. Therefore, the hanging drop array has been formatted to support the growth and interactions of both OvCa cells and macrophages.

CD47-SIRPα signaling has been targeted mainly by anti-CD47 antibodies used in combination with other checkpoint blockades to enhance anticancer function. The main obstacles in these trials include precise delivery and off-target effects, such as autoimmunity risks. The targeted lipid nanoparticle (LNP)-based compositions with siRNAs to specifically inhibit the CD47-SIRPα pathway for metastatic OvCa therapy address these limitations of CD47-SIRPα therapy. In certain embodiments, these LNP not only prevent cargo degradation in transport but can also be preferentially taken up by the phagocytic cells like macrophages, thus specifically targeting these cells.

Embodiments include methods for treating a metastatic tissue by contacting the metastatic tissue with the nanoparticle composition containing the SIRPα siRNA. In certain embodiments, the tissue is a metastatic ovarian cancer. In certain embodiments, the metastatic ovarian cancer presents in the liver. Embodiments of these methods can also include contacting the tissue with a platinum-based chemotherapeutic agent. The platinum-based chemotherapeutic agent may be one or more of cisplatin, carboplatin, oxaliplatin, and picoplatin. In certain embodiments, the metastatic tissue is concurrently contacted with the nanoparticle composition containing the SIRPα siRNA and the platinum-based chemotherapeutic agent. Embodiments of these methods can also include contacting the tissue with an anti-CD47 antibody along with the nanoparticle composition containing the SIRPα siRNA.

Embodiments include methods for treating a subject diagnosed as having ovarian cancer by administering to the subject a therapeutically effective amount of the nanoparticle composition containing the SIRPα siRNA. In certain embodiments, the nanoparticle is a lipid-based nanoparticle. In certain embodiments, the nanoparticle contains a lipid with a preference for localization in a liver. These lipids can be ionizable lipids or cationic lipids. In certain embodiments, the SIRPα siRNA includes sense and antisense sequences of SEQ ID NO. 1 and SEQ ID NO. 2. In certain embodiments, the SIRPα siRNA includes sense and antisense sequences of SEQ ID NO. 3 and SEQ ID NO. 4. In certain embodiments, the ovarian cancer is concomitant with metastasis. Embodiments of these methods can also include administering to the subject a platinum-based chemotherapeutic agent. The platinum-based chemotherapeutic agent may be one or more of cisplatin, carboplatin, oxaliplatin, and picoplatin. In certain embodiments, the methods includes administering to the subject the nanoparticle composition containing the SIRPα siRNA and carboplatin. In certain embodiments, the methods includes administering to the subject concurrently the nanoparticle composition containing the SIRPα siRNA and the platinum-based chemotherapeutic agent. Embodiments of these methods can also include administering to the subject an anti-CD47 antibody along with the nanoparticle composition containing the SIRPα siRNA.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Embodiments will be readily understood by the following detailed description in conjunction with the accompanying drawings. Embodiments are illustrated by way of example and not by way of limitation in the figures of the accompanying drawings.

FIG. 1A is a set of phase contrast micrographs of OvCa or OvCa/macrophage spheroids, under control untreated or carboplatin treatment. FIG. 1B is a graphical representation of the cell viability when treated with varying doses of carboplatin.

FIG. 2A is a set of scanning electron micrographs of fresh porcine livers, that are decellularized to engineer liver biomatrix. Scale=50 μm. FIG. 2B is a graphical representation of the DNA quantification showing removal of all native porcine DNA.

FIG. 3A is an illustration of the macrophage checkpoint CD47-SIRPα axis and FIG. 3B presents the immunohistochemistry of a patient sample with expression of CD47-SIRPα in the ovarian tumor.

FIG. 4 is an illustration of the macrophage checkpoint CD47-SIRPα axis disrupted by siSIRPα-LNP.

FIG. 5A is a set of micrographs of patient-derived OvCa and OvCa/MP spheroids and FIG. 5B is the associated graphical representation of the flow analysis for CD47-SIRPα.

FIG. 6A is a set of micrographs of OvCa/MP hetero-spheroids treated with 500 μM of carboplatin in control (left) or with siSIRPα-LNP treatment (right). Knockdown of SIRPα in hetero-spheroids resulted in increased cell death (indicated by arrows, where there is a loss of spheroid boundary) and sensitization to carboplatin upon viability analysis. Scale bar=200 μm. FIG. 6B is a graphical representation of the normalized cell viability in response to the carboplatin dose.

FIG. 7 is a set of scanning electron (top; scale=50 μm) and multiphoton micrographs (bottom; scale=150 μm) of OCLM from OvCa (top and bottom, left) or OvCa/MP spheroids (top and bottom, right) from OVCAR3/THP1. OvCa/MP OCLM have more metastatic nests within the liver biomatrix (arrows in the bottom panels).

FIG. 8 is a graphical representation of the multiphoton micrographs in untreated (orange) or siSIRPα-LNP treated (teal) OCLM. Quantitatively, siSIRPα-LNP treatment reduces the invasion of OCLM.

FIGS. 9A-9D demonstrate CD47-SIRPα presence in ovarian cancer. (A) Kaplan-Meier survival curve generated from UCSC Xena Browser. Median survival for patients with SIRPα expression in the top 10% decreases by 1.5-fold (p=0.25). (B) Immunohistochemistry staining of primary patient OvCa tumor samples. Brightfield images show brown color change in the slides indicating the presence of CD47 (left) and SIRPα (right) at 20× and 40× magnifications. Scale bars=50 μm. (C) Phase contrast micrographs of spheroids generated from 50 OVCAR3 cells and 50 THP-1-derived M0 macrophages. By day 2, cells began to aggregate (left). Cells proliferated to form a compact spheroid by day 4 (right). Scale bars=200 μm. (D) Analysis of CD47 and SIRPα expression in OvCa/macrophage heterospheroids. Flow cytometry (left) indicates 77.05±8.76% of cells within the spheroids express CD47 and 3.99+0.47% express SIRPα. Western blotting confirmed the presence of the SIRPα protein in the heterospheroids (right) validating the maintenance of a macrophage population within the spheroid model.

FIGS. 10A-10C demonstrate macrophage-dependent chemoresistance in 3D spheroids. FIG. 10A is a set of phase contrast micrographs of monospheroids (OvCa cells alone) and heterospheroids (OvCa/macrophage coculture). Comparison of day 1 and 4 images shows the cells clustering at the bottom of the hanging drop and forming a single compact spheroid by day 4. Mono- and heterospheroids were seeded with 100 cells/drop (day 0) but monospheroids appear larger by day 4. Compact spheroids were treated with carboplatin chemotherapy (0-500 μM) on day 4, and visualized at Day 6. Cell death was evident by increased cellular debris surrounding the spheroid as well as less defined spheroid and cell boundaries. FIG. 10B is a graphical representation of the proliferation measured by MTS viability assay confirmed the increased sizes observed in monospheroids compared to heterospheroids in spheroid images. Monospheroids (black trace) grew nearly 2.4 times more than heterospheroids (orange trace) by day 6 owing to the terminal differentiation state of macrophages which inhibited their proliferation. FIG. 10C is a graphical representation of the monospheroid and heterospheroid response to carboplatin chemotherapy (0-500 μM) was measured with an MTS viability assay. Heterospheroids were more resistant with an IC₅₀ value of 368.2 μM compared to monospheroids (218.1 μM).

FIGS. 11A-11E present LNP design and characterization. LNP were fabricated using microfluidic techniques to encapsulate siSIRPα and were characterized for stability and consistency. FIG. 11A is a graphical representation of the size distribution and polydispersity index of siSIRPα LNP from three separate prepared batches. These siSIRPα LNP measured around 60 nm in diameter with low polydispersity index (PDI, <0.1) indicating fabrication consistency both within and between batches of particles as measured by dynamic light scattering (DLS, left). Size distribution plots also demonstrate the desired consistency in particle diameter (right). FIG. 11B is a graphical representation of the DLS analysis that showed siSIRPα LNP were neutrally charged with zeta potential averaging around −4 mV (left) with consistent distribution between batches (right). FIG. 11C is a graphical representation of the encapsulation efficiency of the siSIRPα LNP. The amount of siRNA encapsulated within the particles remained highly consistent between batches (left) with >95% efficiency of encapsulating siRNA in all batches (right). FIG. 11D is a transmission electron micrograph (TEM) of siSIRPα LNP. Particle size distribution can be visualized in the TEM taken at 20,000× magnification. Scale bar=200 nm. FIG. 11E is a set of graphical representations of the particle size/PDI (left), zeta potential (middle) and encapsulation (right), which demonstrate that the siSIRPα LNP remain consistent over 10 days indicating the stability of the siSIRPα LNP for the duration of experiments.

FIGS. 12A-12C demonstrate the siSIRPα LNP uptake and alteration of macrophage phenotype. FIG. 12A on the left presents the live-cell imaging of M0 macrophages in 2D culture. Fluorescently-labeled LNP containing SIRPα siRNA were added to macrophages and monitored over 48 hours with hourly imaging. LNP uptake is indicated by red fluorescence which visibly increased over time as more macrophages phagocytosed the LNP (FIG. 12A, left). FIG. 12A on the right is a graphical representation of the percentage of fluorescent cells over time. Analysis of hourly images over 48 hours showed more than 50% of cells uptake the LNP by 30 hours (FIG. 12A, right). FIG. 12B is a graphical representation of the gene expression analysis indicating that macrophages treated with siSIRPα LNP expressed 24.28±1.34% less SIRPα than untreated controls. (****p<0.0001, unpaired t test). FIG. 12C is a graphical representation of the polarization of the M0 macrophages towards an MI phenotype. Addition of siSIRPα LNP to M0 macrophages induced a shift toward an anti-tumoral (M1) phenotype defined by a 1.54±0.14-fold increase in IL-12 expression and a corresponding 0.33±0.12-fold decrease in IL-10 expression.

FIGS. 13A-13C present the siSIRPα LNP uptake by M2 macrophages. The immunosuppressive nature of the OvCa tumor microenvironment can drive macrophage polarization toward alternative activation. FIG. 13A presents the live-cell imaging of THP-1-derived macrophages that were polarized to M2-like phenotypes with MCSF and IL-4. M2 macrophages efficiently took up fluorescently labeled siSIRPα LNP as indicated by red signal in live cell images taken over 48 hours after LNP administration. Uptake continued over 48 hours with increased red fluorescence visible with time. FIG. 13B is a graphical representation of the image quantification confirmed this uptake efficiency showing that >50% of cells exhibited fluorescence by 30 hours. FIG. 13C is a graphical representation of the polarization of the M2 macrophages towards an M1 phenotype. Following confirmation of particle uptake, M2 macrophages treated with siSIRPα LNP were evaluated for LNP ability to shift polarization. LNP did induce a more M1-like gene signature with a significant increase in IL-12 and a corresponding reduction in IL-10 M2 expression (***p<0.001 ). This indicated the ability to reverse the pro-tumoral activation state commonly expressed by macrophages in the OvCa tumor microenvironment.

FIGS. 14A-14E demonstrate that reducing SIRPα reduces metastasis-indicating behavior in OvCa/macrophage heterospheroids. FIGS. 14A and 14B are graphical representations of the effects of siSIRPα LNP treatment of heterospheroids. This treatment resulted in a reduction of nearly 42% (****p<0.0001) of SIRPα gene expression (FIG. 14A, left) which translated to a similar 50% reduction in protein expression (FIG. 14A, right). This decrease in SIRPα expression was linked to a recovery of chemotherapy response in resistant heterospheroids (FIG. 14B). In fact, siSIRPα LNP induced a 32% recovery of the resistance caused by macrophages as compared to OvCa monospheroid controls. FIG. 14C is a set of phase contrast micrographs of control and LNP-treated heterospheroids. Images showed slowed growth by day 2 following LNP treatment. LNP-treated heterospheroids were more sensitive to carboplatin chemotherapy, showing increased cell death by day 4 across 5 doses used to generate an IC₅₀ value. FIG. 14D is a graphical representation of the metastasis-indicating gene analysis of OvCa cells. Cells from heterospheroids were treated with siSIRPα LNP or maintained as controls and sorted to analyze OvCa cells alone. Both heterospheroid conditions were compared to macrophage-naïve OVCAR3 cells from monospheroids. Loss of MMP-9 gene expression was observed after siSIRPα LNP (compare 2.37-fold 2.85-fold increase in MMP-9 in control heterospheroids to 2.37-fold after LNP, ***p=0.0005, two-way ANOVA) which indicated OvCa cells becoming less invasive following LNP treatment. Similarly, there is a small decrease in epithelial-to-mesenchymal transition gene SNAI1. FIG. 14E on the left is a set of photographs showing the spheroid migration model created to measure OvCa invasiveness. Spheroids were maintained in hanging drop culture for 4 days with siSIRPα LNP treatment on day 2. After seeding onto 2D well-plates, cellular migration was tracked via changes in area occupied by cells as they traveled away from the spheroid as indicated by the colored outlines drawn in ImageJ software (left). FIG. 14E, right panel, is a graphical representation of the migratory ability. Macrophage presence induced a significant increase in migratory ability (****p<0.0001, two way ANOVA) which was partially reversed in spheroids which were pre-treated with LNP prior to seeding (right).

FIGS. 15A-15D present the carboplatin sensitivity in control spheroids. siSIRPα LNP treatment was shown to slow growth and decrease viability in chemotherapy-treated heterospheroids. To confirm whether this was a toxic effect of the LNP or rather OvCa growth reduction resulting from CD47-SIRPα inhibition, additional controls were tested in the presence of LNP. FIG. 15A is a graphical representation of the cell viability in the OvCa monospheroids in response to carboplatin with and without siSIRPα LNP treatment. OvCa monospheroids were similarly responsive to carboplatin with and without siSIRPα LNP treatment (compare IC₅₀ value of 218.1 μM in control spheroids to 272.1μMafter siSIRPα LNP). As hypothesized, LNP did not appear to have a toxic effect on the OvCa cells as evidenced by this maintenance of cell viability. FIG. 15B is a set of associated phase contrast micrographs that show comparable spheroid size and morphology after 2 days of LNP treatment and after another 2 days with 0-500 μM chemotherapy confirming the LNP are not acting on OvCa cells to reduce viability.

FIG. 15C is a graphical representation of the cell viability in the OvCa monospheroids in response to carboplatin with and without control LNP containing negative control (scramble) siRNA. These LNPs were administered to OvCa/macrophage heterospheroids to evaluate the potential adverse effects of the LNP vehicle on spheroid viability. A transmission electron micrograph (50,000×; scale bar=50 nm) illustrates the uniform size distribution of negative control LNP (insert in FIG. 15C). FIG. 15D is a set of associated phase contrast micrographs. Heterospheroids did not exhibit any signs of toxicity in response to the negative control LNP as evidenced by similar IC₅₀ values (368.2μM with no treatment and 405.3 μM with scramble LNP) and phase contrast micrographs (scale bar=200 μm).

FIGS. 16A-16B are graphical representations of the ID8/C57 IVIS luminescence imaging of both the whole body (FIG. 16A) and abdomen (FIG. 16B) at the end point. Data here shows the decreased tumor burden in the LNP carrying SIRPα siRNA (5 μg)+carboplatin (1.0 mg/kg) group, as the cells are tagged with luminescent luciferase.

FIGS. 17A-17C present Ki67 staining in individual lung, spleen, liver, and kidney tissue of mice treated with SIRPα siRNA (5 μg)+Carboplatin (1.0 mg/kg) when compared to mice treated with 1×PBS. FIG. 17D is a graphical representation of the Ki67 staining in the total aggregate expression in organs of mice treated with SIRPα siRNA (5 μg)+Carboplatin (1.0 mg/kg) when compared to mice treated with 1×PBS.

FIG. 18 is a graphical representation of the Ki67 IHC staining analysis of all organs that present significant lowering of the proliferation marker with treatment group siSIRPα LNP (5 μg)+carboplatin (1.0 mg/kg).

### DETAILED DESCRIPTION

Most ovarian carcinoma (OvCa) patients present with advanced disease at the time of diagnosis. Malignant, metastatic OvCa is chemoresistant and invasive with poor prognosis, exposing the need for improved therapeutic targeting. High CD47 (OvCa) and SIRPα (macrophage) expression have been linked to decreased survival, making this interaction a significant target for therapeutic discovery. Macrophages are an important component of the OvCa tumor microenvironment and are manipulated to aid in cancer progression via CD47-SIRPα signaling. CD47-SIRPα therapies have been historically limited by specificity. Disclosed herein are nanoparticles that target phagocytic macrophages expressing the SIRPα protein in metastatic tissues. Certain embodiments include lipid-based nanoparticles (LNP) containing siRNAs that target the SIRPα expression in metastatic tissues.

Provided here are nanoparticle compositions containing siRNA to disrupt the signal regulatory protein-α (SIRPα) signaling pathway. In an embodiment, this disrupts the signaling pathway between ovarian cancer cells and macrophages to treat advanced ovarian cancer and ovarian cancer metastasis. In certain embodiments, the nanoparticle composition is a lipid nanoparticle. In certain embodiments, the nanoparticle composition is a liposome. Embodiments include a pharmaceutical composition with a therapeutically effective amount of the nanoparticle composition containing SIRPα siRNA. Embodiments of the SIRPα siRNA include sequences presented as sense and antisense sequences that target one or more portions of the signal regulatory protein-a gene. Embodiments of the SIRPα siRNA include sequences presented as sense and antisense sequence of SASI_Hs01_00145338 that target the gene region starting at position 1458, provided below as SEQ ID NO. 1 and SEQ ID NO. 2.

| | Sense sequence (5′-3′): |
|---|---|
| SEQ ID NO. 1 | |
| | CUAAUGAACGGAACAUCUA[dT][dT] |
| | Antisense sequence (5′-3′): |
| SEQ ID NO. 2 | |
| | UAGAUGUUCCGUUCAUUAG[dT][dT] |

Embodiments of the SIRPα siRNA include sequences presented as sense and antisense sequence of SASI_Hs01_00017994 that target the gene region starting at position 1366, provided below as SEQ ID NO. 3 and SEQ ID NO. 4.

| | Sense sequence (5′-3′): |
|---|---|
| SEQ ID NO. 3 | |
| | CUAAUGAACGGAACAUCUA[dT][dT] |
| | Antisense sequence (5′-3′): |
| SEQ ID NO. 4 | |
| | UAGAUGUUCCGUUCAUUAG[dT][dT] |

Embodiments include methods for treating a metastatic tissue by contacting the tissue with the nanoparticle composition containing SIRPα siRNA. In certain embodiments, the metastatic tissue is an ovarian cancer tissue. In certain embodiments, the metastatic ovarian cancer presents in the liver. Embodiments of these methods can also include contacting the tissue with a platinum-based chemotherapeutic agent. The platinum-based chemotherapeutic agent may be one or more of cisplatin, carboplatin, oxaliplatin, and picoplatin. In certain embodiments, the metastatic tissue is concurrently contacted with the nanoparticle composition containing the SIRPα siRNA and the platinum-based chemotherapeutic agent. In certain embodiments, the metastatic tissue is first contacted with the nanoparticle composition containing the SIRPα siRNA, and subsequently the platinum-based chemotherapeutic agent. In certain embodiments, the metastatic tissue is partially or completely resistant to a platinum-based chemotherapeutic agent prior to contact with the nanoparticle composition containing the SIRPα siRNA. Embodiments of these methods can also include contacting the tissue with an anti-CD47 antibody along with the nanoparticle composition containing the SIRPα siRNA.

Embodiments include methods for treating a subject diagnosed as having ovarian cancer by administering to the subject a therapeutically effective amount of the nanoparticle composition containing the SIRPα siRNA. In certain embodiments, the nanoparticle is a lipid-based nanoparticle. In certain embodiments, the nanoparticle contains a lipid with a preference for localization in a liver. In certain embodiments, the SIRPα siRNA includes sense and antisense sequences of SEQ ID NO. 1 and SEQ ID NO. 2. In certain embodiments, the SIRPα siRNA includes sense and antisense sequences of SEQ ID NO. 3 and SEQ ID NO. 4. In certain embodiments, the ovarian cancer is concomitant with metastasis. Embodiments of these methods can also include administering to the subject a platinum-based chemotherapeutic agent. The platinum-based chemotherapeutic agent may be one or more of cisplatin, carboplatin, oxaliplatin, and picoplatin. In certain embodiments, the methods includes administering to the subject concurrently the nanoparticle composition containing the SIRPα siRNA and the platinum-based chemotherapeutic agent. In certain embodiments, the methods includes administering to the subject the nanoparticle composition containing the SIRPα siRNA and then the platinum-based chemotherapeutic agent. In certain embodiments, the ovarian cancer in the subject is partially or completely resistant to a platinum-based chemotherapeutic agent prior to administration of the nanoparticle composition containing the SIRPα siRNA. Embodiments of these methods can also include administering to the subject an anti-CD47 antibody along with the nanoparticle composition containing the SIRPα siRNA

CD47-SIRPα presence was evaluated in patient histological sections using immunohistochemistry. 3D tumor spheroids were generated on a hanging drop array with OVCAR3 high-grade serous OvCa and THP-1-derived macrophages, creating a representative model of cellular interactions involved in metastatic OvCa. Microfluidic techniques were employed to generate LNP encapsulating SIRPα siRNA (siSIRPα) to target CD47-SIRPα signaling between OvCa and macrophages. siSIRPα LNP were characterized for optimal size, charge and encapsulation efficiency. Uptake of siSIRPα LNP in THP-1 derived macrophages were assessed by Incucyte® Live-Cell Analysis System. Following 48 hours of 25 nM siSIRPα treatment, OvCa/macrophage heterospheroids were evaluated for SIRPα knockdown, platinum chemoresistance (via cell viability), and invasive potential.

OvCa patient tumors as well as OvCa/macrophage heterospheroids expressed both CD47 and SIRPα. Macrophages in OvCa spheroids increased carboplatin resistance and invasion indicating a more malignant phenotype. Successful uptake of LNP by macrophages was observed causing significant reduction in SIRPα gene expression and subsequent reversal of pro-tumoral alternative activation. Blocking CD47-SIRPα signaling in heterospheroids also resulted in reduced SIRPα protein expression. Disrupting CD47-SIRPα interactions in heterospheroids resulted in sensitizing OvCa/macrophage heterospheroids to platinum chemotherapy, and reversal of cellular invasion outside of heterospheroids. Thus, the LNP-based therapy can reduce malignant progression of ovarian cancer.

Provided herein are compositions and methods for treatment of ovarian cancer (OvCa) metastases infiltrating the liver and peritoneum. These compositions directly impact patient survival and quality of life with targeted therapeutics and improve parity in cancer treatment. The average 5-year survival rate for metastatic ovarian cancer is a mere 30%, thereby requiring new therapeutic intervention strategies that can improve survivorship. Methods of treatment include the use of targeted LNP to unlock immune checkpoint signaling that drives ovarian cancer metastases. These LNP nanotherapeutic compositions disrupt macrophage based chemoresistance and infiltrative metastasis of OvCa.

An “effective amount” or “therapeutically effective amount” is an amount sufficient to effect desired results (such as desired clinical results, to achieve therapeutic efficacy). A therapeutically effective dose can be administered in one or more administrations and can vary depending on the compound, the disease and its severity, and the age, weight, etc., of the subject to be treated.

“Administering” refers to the physical introduction of a therapeutic agent to a subject in need thereof. Exemplary routes of administration for LNPs include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. Administering can also be performed, for example, once, a plurality of times, and/or over one or more extended periods. LNPs containing the siRNA can be constituted in a composition, such as a pharmaceutical composition containing the LNP and a pharmaceutically acceptable carrier. As used herein, a “pharmaceutically acceptable carrier” includes any and all solvents, dispersion media, coatings, antibacterial and antifungal agents, isotonic and absorption delaying agents, and the like that are physiologically compatible.

“Treating” or “treatment” of any disease or disorder refers, in one embodiment, to any indicia of success in the amelioration of an injury, disease, or condition, including any objective or subjective parameter such as abatement, remission, diminishing of symptoms or making the injury, disease, or condition more tolerable to the subject, slowing in the rate of degeneration or decline, making the final point of degeneration less debilitating, and/or improving a subject's physical or mental well-being. In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both.

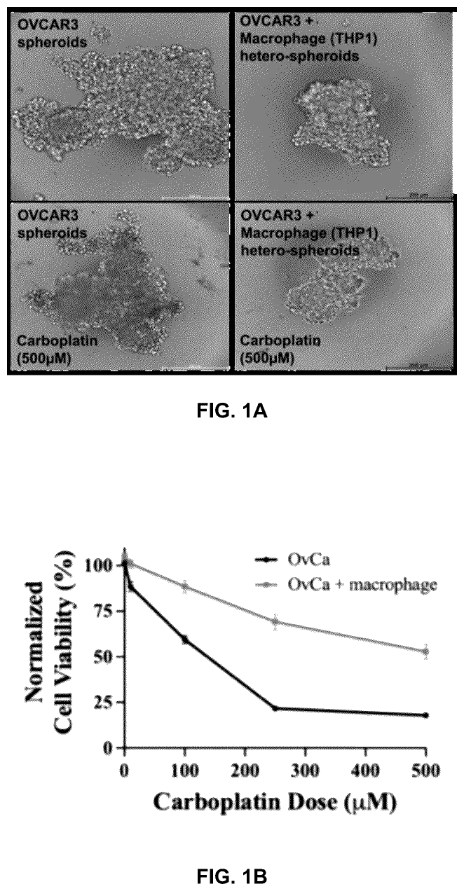

High grade serous OvCa metastasizes directly to abdominal cavity organs based on its unique propensity to direct seeding and bypassing traditional extravasation/intravasation barriers that other types of solid cancers use. Although hematogenous spread has been described in OCLM, primary OvCa tumors shed metastatic cells directly into the ascites fluid, aggregate into spheroids, and get transported throughout the peritoneal cavity directly seeding into secondary organs such as the omentum and liver. Within the ascites, OvCa cells interact with several stromal cells, including macrophages. The 3D OvCa spheroids can be derived from malignant patient ascites, and from OvCa cell lines. Inclusion of macrophages within these spheroids can create OvCa/Macrophage hetero-spheroids, that are chemoresistant and metastatic (FIG. 1A). FIG. 1A is a set of phase contrast micrographs of OvCa or OvCa/macrophage spheroids, under control untreated or carboplatin treatment. Scale32 200 μm. FIG. 1B is a graphical representation of the cell viability when treated with varying doses of carboplatin. Viability analysis indicated that OvCa/macrophage spheroids were significantly more chemoresistant to carboplatin, with higher IC₅₀.

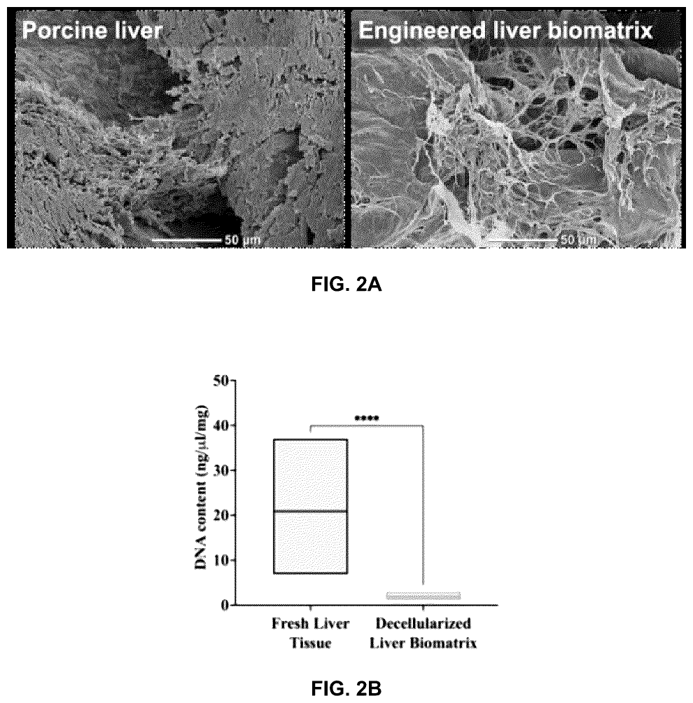

Embodiments include use of an established hanging drop array spheroid platform to initiate metastatic OvCa clusters that seed into the liver. These hanging drop array spheroid platforms can serve as research models or screening tools for therapeutic agents. These models are advantageous because in vitro studies of invasive and malignant cell behaviors rely on outdated models like scratch/wound healing assays or Transwell assays. Tumor cell clustering and invasion are inherently 3D processes, which rely on native tissue orientation and architecture. Cellular responses to these stimuli are often lost in the 2D setting, limiting predictive capability. Hydrogels can partially mimic 3D cell interactions with the benefit of tunable properties, such as stiffness and porosity, but lack native tissue extracellular matrix (ECM) influence. ECM proteins extracted from tissues and reconstituted to fabricate 3D scaffolds, while better than hydrogels, are still limited by their lack of native ECM architecture. Therefore, models limiting these interactions will underperform when used to study cell behaviors, such as immune signaling patterns and metastatic outgrowth. Disclosed herein are novel liver biomatrix scaffolds that maintain liver ECM 3D architecture and support the growth of metastatic nests. These liver biomatrix scaffolds are engineered by decellularization of porcine livers and support the 3D invasion, and colonization of cancer cells retaining dependency on protease activity (FIGS. 2A and 2B). FIG. 2A is a set of scanning electron micrographs of fresh porcine livers (FIG. 2A, left), that are decellularized to engineer liver biomatrix (FIG. 2A, right). Scale=50 μm. FIG. 2B is a graphical representation of the DNA quantification showing removal of all native porcine DNA.

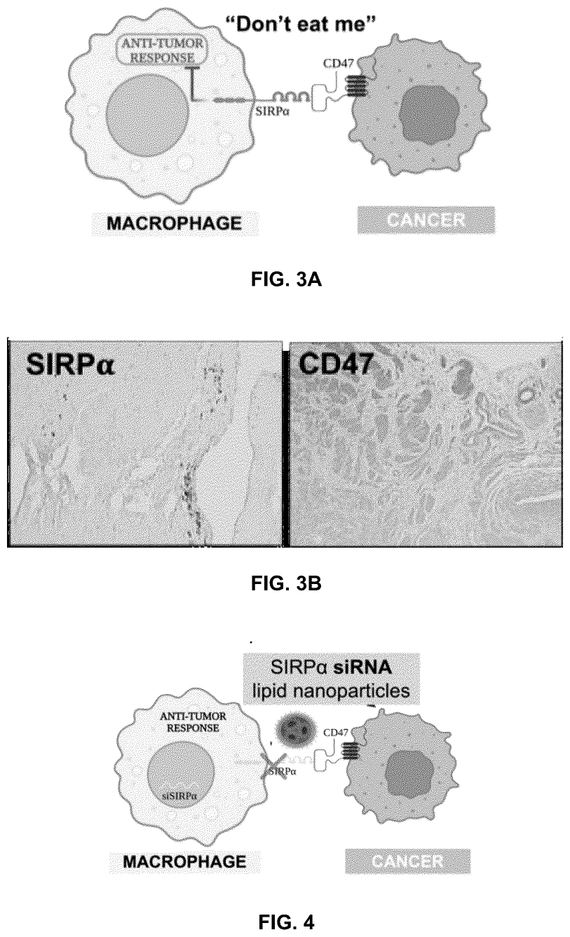

Macrophages drive OvCa invasion, and chemoresistance through reciprocal Wnt signaling. Macrophages and their immune checkpoint signaling drive OvCa invasion and chemoresistance in 2D models. SIRPα is an important transmembrane checkpoint receptor expressed on macrophages responsible for recognizing its ligand CD47 on normal cells. Upon CD47-SIRP binding, cytoplasmic domains of SIRPα are phosphorylated, inhibiting macrophage phagocytosis. Normal cells expressing the self-antigen CD47 can send a “Don't Eat Me” signal, allowing macrophages to recognize healthy cells, thereby maintaining homeostasis (FIGS. 3A-3B). FIG. 3A is an illustration of the macrophage checkpoint CD47-SIRPα axis and FIG. 3B presents the immunohistochemistry of a patient sample with expression of CD47 (FIG. 3B, right) and SIRPα (FIG. 3B, left) in the ovarian tumor. OvCa cells abnormally express the CD47 protein, allowing them to leverage this signaling pathway to evade immune cell killing. This overexpression is associated with increased migration and invasion at the cellular level, and worse prognosis in the clinic, indicating the CD47/SIRPα signaling axis is a relevant therapeutic target. CD47/SIRPα signaling has been targeted mainly by anti-CD47 antibodies used in combination with other checkpoint blockades to enhance anticancer function. Main obstacles in these trials include precise delivery and off-target effects, including pancytopenia and neurologic adverse events. To overcome these limitations, novel nanoparticle based delivery of SIRPα targeting agents has been developed that can disrupt CD47-SIRPα signaling between OvCa and macrophages in the liver metastatic microenvironment. In certain embodiments, the nanoparticles are lipid-nanoparticles.

Development of efficacious chemotherapeutic strategies in OCLM is hampered by the complexity of the OCLM microenvironment. The liver has a dense network of capillaries, or sinusoids, reaching the innermost cells in the organ efficiently providing oxygen and soluble nutrients. Therefore, metastases growing in the liver preserve the stromal structure of the liver and do not rely on angiogenesis for survival. Tumor cells in the liver primarily use existing vasculature of the surrounding parenchyma. Liver metastases are characterized by poor permeation of molecules, where even intravenously injected contrast agents do not permeate. This unfavorable diffusion pattern is an important factor limiting adequate concentration of therapeutics and could explain why chemotherapy fails to cure liver lesions. The lack of efficient transport into cancer lesions within the liver tissue become even more challenging with large molecules, as with microaggregated albumin. This pattern of a highly vascularized liver tissue hosting hypovascularized tumors is conducive of poor therapeutic responses and higher mortality.

Inflammation in the liver metastatic sites attracts numerous immune cells, primarily macrophages, which are professional phagocytes. Nanomedicines naturally hone to the macrophages in the liver even when systemically administered. Targeting macrophages with nanocarriers in breast and lung liver metastasis can polarize macrophages from a pro-tumorigenic (M2) phenotype to anti-tumor M1 macrophages. Compositions disclosed herein take advantage of the affinity of the nanoparticles to the macrophages for siRNA-based macrophage checkpoint immunotherapy. Previously, viral transduction and DNA plasmids transfection have been extensively used as gene therapy methods in vivo. However, these delivery approaches may integrate and permanently alter the human genome and, therefore, have not been translated to the clinic.

Compositions include SIRPα siRNA-LNP (siSIRPα-LNP) that target macrophages in the OCLM, making the lesions susceptible to platinum chemotherapy. FIG. 4 is an illustration of the macrophage checkpoint CD47-SIRPα axis disrupted by siSIRPα-LNP. Affecting this immune axis in advanced OvCa disease will impact therapeutic benefits for women with advanced cancers.

OvCa cells are shed from the primary tumor, into the malignant ascites, and interact with macrophages as floating spheres. The engineered hanging drop array spheroids mimic the interaction of OvCa and macrophages (MP) within the malignant ascites-in this model and in vivo, macrophages drive adaptive resistance to immunotherapy and metastasis. In this interaction context, CD47-SIRPαpromotes OvCa cell metastasis, and cell growth, making it a relevant therapeutic axis. Physico-chemical LNP properties (size, surface charge, etc.) affect their biodistribution and ability to target the liver.

Embodiments of the compositions disclosed herein include lipids that specifically localize to liver (for example, ionizable lipids, such as Dilinoleyl-methyl-4-dimethylaminobutyrate MC3) as well as lipids that produce immunological reactions, aiming at a possible “adjuvant” effect (for example, cationic lipids, such as DOTAP) with macrophage checkpoint inhibitors. Other ionizable lipids include and are not limited to [(4-Hydroxybutyl)azancdiyl]di(hexane-6,1-diyl) bis(2-hexyldecanoatc) ALC-135; ALC-0315, BP Lipid 216; BP Lipid 217 (CAS 2430034-17-4); Lipid III-45 (CAS 2096984-25-5); BP Lipid 226 (CAS 2036272-94-1); SM-102 (CAS: 2089251-47-6). Other cationic lipids include and are not limited to DOTMA (CAS 104162-48-3), SM-102 N-oxide (CAS 2824195-50-6), TAP (CAS 197974-74-6, 139984-36-4, 220609-41-6, 144189-73-1).

Ovarian cancer cell/macrophage (OvCa/MP) hetero-spheroids are resistant to carboplatin treatment. Hanging drop array spheroid cultures were generated hetero-spheroids from the high grade serous ovarian cancer cell line, OVCAR3 and THP1 monocyte-derived macrophages and peripheral blood monocyte-derived macrophages (PBMCs). Cells aggregate in hanging drop array cultures over 4 days and form compact spheroids (FIG. 1A). Carboplatin (0-500 μM) treatment of OVCAR3 or OVCAR3/THP1 spheroids resulted in higher IC₅₀ values with macrophages (114 μM in OVCAR3 monospheroids vs. 535.7 μM in OVCAR3/THP1 OvCa/MP; FIG. 1B).

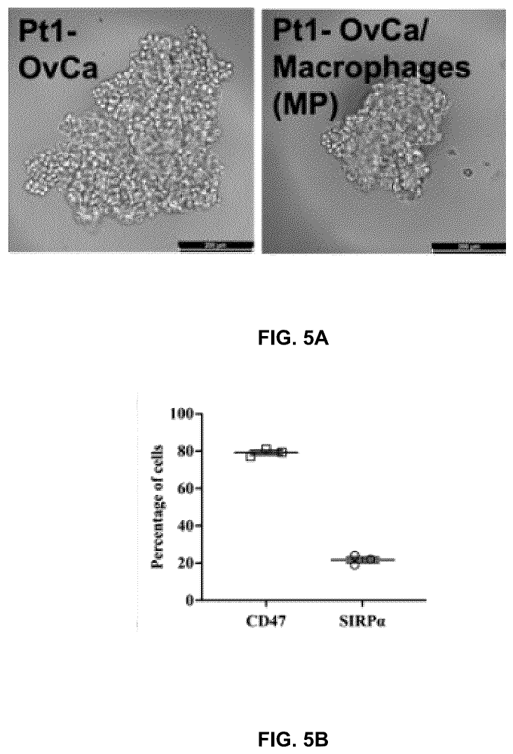

Cell line and patient-derived OvCa/MP hetero-spheroids express robust CD47-SIRPα signaling. Engineered OvCa/MP hetero-spheroids from both cell line, and patient derived samples express CD47 and SIRPα, indicating an active macrophage immune checkpoint. Flow analysis was used to evaluate the expression of the macrophage checkpoint (FIGS. 5A-5B). FIG. 5A is a set of micrographs of patient-derived OvCa and OvCa/MP spheroids and FIG. 5B is the associated graphical representation of the flow analysis for CD47-SIRPα.

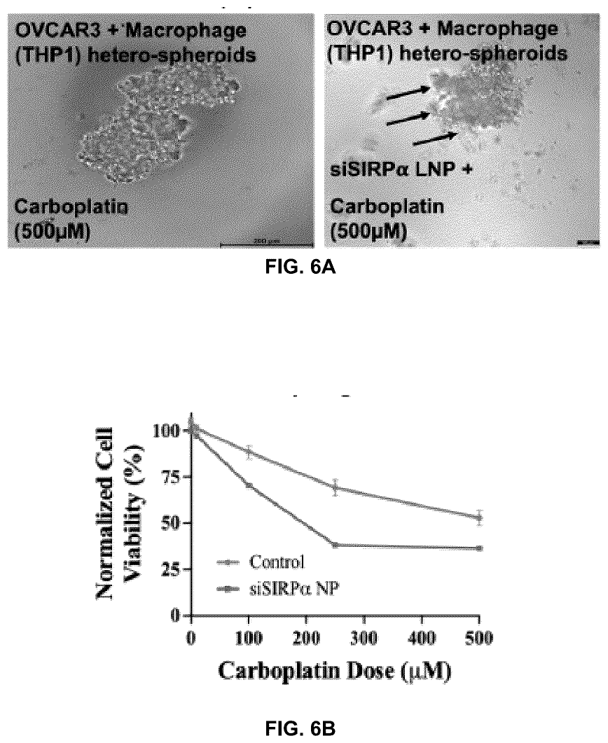

siSIRPα LNP knocks down SIRPα expression in macrophages and improves sensitivity to carboplatin in OvCa/MP hetero-spheroids. The siSIRPα was preselected following rigorous selection of several siRNAs, resulting in >50% knockdown of SIRPα gene and protein expression in macrophages. Cell line-derived OvCa/MP hetero-spheroids were treated with siSIRPα-LNP and robust knockdown of macrophage expression of SIRPα was observed (up to 50% gene knockdown). SIRPα knocked down macrophages reversed carboplatin resistance, shown by the lowered cell viability upon carboplatin treatment and a reduction in IC₅₀ to 213.2 μM from 535.7 μM (FIGS. 6A-6B). FIG. 6A is a set of micrographs of OvCa/MP hetero-spheroids treated with 500 μM of carboplatin in control (left) or with siSIRPα-LNP treatment (right). Knockdown of SIRPα in hetero-spheroids resulted in increased cell death (indicated by arrows, where there is a loss of spheroid boundary) and sensitization to carboplatin upon viability analysis. Scale bar=200 μm. FIG. 6B is a graphical representation of the normalized cell viability in response to the carboplatin dose.

Certain embodiments of the spheroids can include more high grade serous ovarian cancer cell lines (OVSAHO, KURAMOCHI, murine ID8) and/or patient-derived cells from at least 5 independent samples. Embodiments can also include healthy donor peripheral blood monocytes (PBMCs) as well as murine bone-marrow derived macrophages for the ID8 line. Using flow cytometry, the robust presence of CD47 and SIRPα were assessed.

Embodiments can include siSIRPα-LNP compositions, for example with cationic lipids DOTAP and DODMA or with ionizable lipids, Dlin-MC3-DMA and SM-102. Besides the charged lipid, the compositions can contain one or more of dioleoylphosphatidylcholine (DOPC), cholesterol, 1,2-Dimyristoyl-rac-glycerol-3-methoxy polyethylene glycol-2000 (DMG-PEG 2000), and phosphatidylethanolamine (PE). Embodiments includes several lipids and lipid/siRNA ratios to optimize siRNA loading and LNP characteristics. For example, lipids (ethanolic phase) and siRNA (aqueous phase) can be combined using a microfluidic chip at the ratio of 1:3 and a flow rate of 4-10 ml/min. LNP can be labeled with a Cy5.5 probe in the membrane. The mixture can be dialyzed in phosphate-buffered saline for 8 hours at 4° C. to remove ethanol and unbound siRNA. Physico-chemical characteristics of siSIRPα-LNP, including diameter (size) and zeta potential (charge), can be assessed in phosphate buffer 10 mM by Dynamic Light Scattering (DLS) in a Malvern nano-ZS Zetasizer. The siRNA encapsulation efficiency in the LNP can be quantified using RiboGreen® RNA assay (Life Technology, CA, USA), following permeabilization with 1% triton and incubation (5 min, 37° C.) to quantify siRNA inside the LNP. As mentioned above, encapsulation efficiency of siRNA in LNP is >90%. To assess siRNA integrity in LNP High-Resolution Automated Electrophoresis can be performed using the Agilent 2100 Bioanalyzer system. RNase-A digestion/free siRNA can serve as controls.

Provided herein are siSIRPα-LNP formulations that reprogram macrophages and alleviate carboplatin resistance within OvCa/MP hetero-spheroids (generated from several cell or patient-derived lines). These formulations can reduce OvCa/MP proliferation by knocking down macrophage SIRPα expression. Certain methods of treatment include concurrent treatment of LNP and carboplatin.

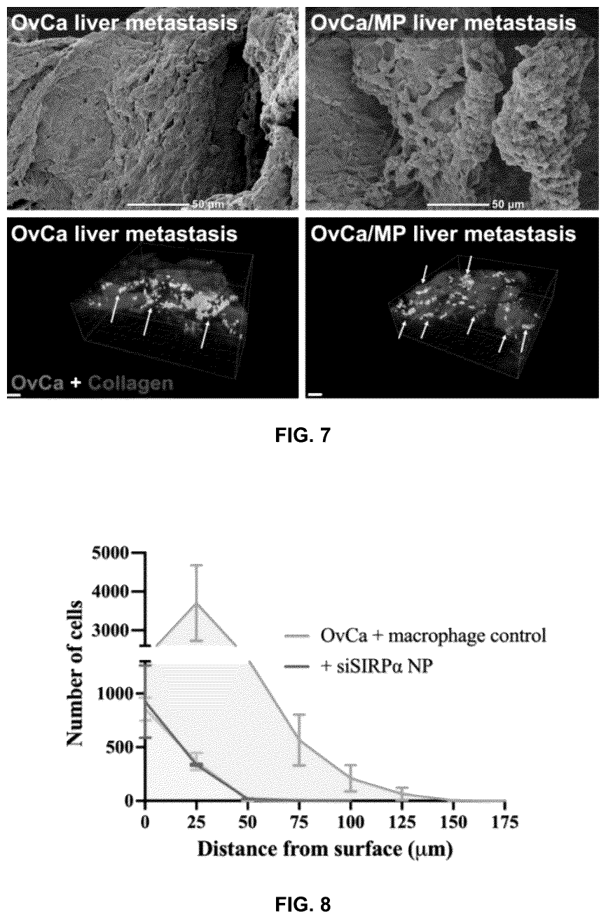

OvCa/MP hetero-spheroids establish OCLM in a decellularized liver biomatrix. When OvCa or OvCa/MP spheroids are seeded onto decellularized porcine liver biomatrix scaffolds, they invade and colonize the biomatrix resulting in engineered OCLM (FIG. 7). FIG. 7 is a set of scanning electron (top; scale=50 μm) and multiphoton micrographs (bottom; scale=150 μm) of OCLM from OvCa (top and bottom, left) or OvCa/MP spheroids (top and bottom, right) from OVCAR3/THP1. OvCa/MP OCLM have more metastatic nests within the liver biomatrix (arrows in the bottom panels). Visually, as observed in both scanning electron micrographs, and multiphoton micrography, GFP tagged OvCa cells invade and colonize the biomatrix more effectively when macrophages are present (number of white arrows; FIG. 7). This is evident by the distributed colonization of green fluorescent cells within the red biomatrix in OvCa/MP hetero-spheroids, compared to a more localized pattern of green cells in OvCa monospheroids. OvCa or OvCa/MP hetero-spheroids (cell lines and patient-derived) can be seeded peri-hepatically on the liver biomatrix, to simulate malignant ascites.

The efficacy of the various siSIRPα-LNP delivery to OCLM on invasion and carboplatin resistance can be evaluated as described herein. The efficacious siSIRPα LNP formulations with improved carboplatin sensitivity can be engineered. OCLM can be established using OvCa or OvCa/MP and treated with siSIRPα-LNP (25-100 nM siRNA) or controls. Following 24-72 hours after LNP treatment, the viability of established nests in response to carboplatin (0-800 μM) for 48 hours is evaluated. In addition to carboplatin sensitivity, multiphoton microscopy can be used to orthogonally validate metastatic nest size of treated/untreated OCLM, as well as identify if siSIRPα LNP can reduce invasion of OCLM. The 50 nM treatment of siSIRPα LNP in OvCa/MP OCLM reduces the invasion of ovarian cancer cells into the biomatrix (quantified as the number of cells invaded from the surface of the biomatrix). FIG. 8 is a graphical representation of the multiphoton micrographs in untreated (orange) or siSIRPα-LNP treated (teal) OCLM. Quantitatively, siSIRPα-LNP treatment reduces the invasion of OCLM.

An increased resistance to carboplatin in OCLM established with OvCa/MP is expected, corroborative to increased invasion mediated by macrophages. Currently, macrophages in the OCLM model are derived from seeding them from OvCa/MP hetero-spheroids. In certain embodiments, the biomatrix can be pre-populated with macrophages. This approach may be advantageous in modeling the presence of macrophages in the precancerous liver niche, prior to OCLM infiltration. The siSIRPα-LNP treatment can mitigate extent of OCLM and sensitize to carboplatin. LNP transport into the biomatrix can be visualized with fluorescent labeling of LNP.

Embodiments include LNPs with >90% encapsulation efficiency of siRNA and a diameter within the range of 60-120 nm. In certain embodiments, the LNPs can be positively charge LNPs and absorb the negatively charged siRNA on the surface. Based on the sensitivity of naked siRNA to enzymatic and chemical degradation and the properties of LNP in protecting siRNA integrity, gene silencing can be at least 2.5 higher in the siSIRPα-LNP group compared to the naked siRNA group. Embodiments include siSIRPα-LNP formulations that effectively curb OCLM, reprogram macrophages within the liver to increase platinum sensitivity. These formulations may be effective against at least 60% of cell and patient-derived lines. In certain embodiments, the LNP formulations are cGMP grade.

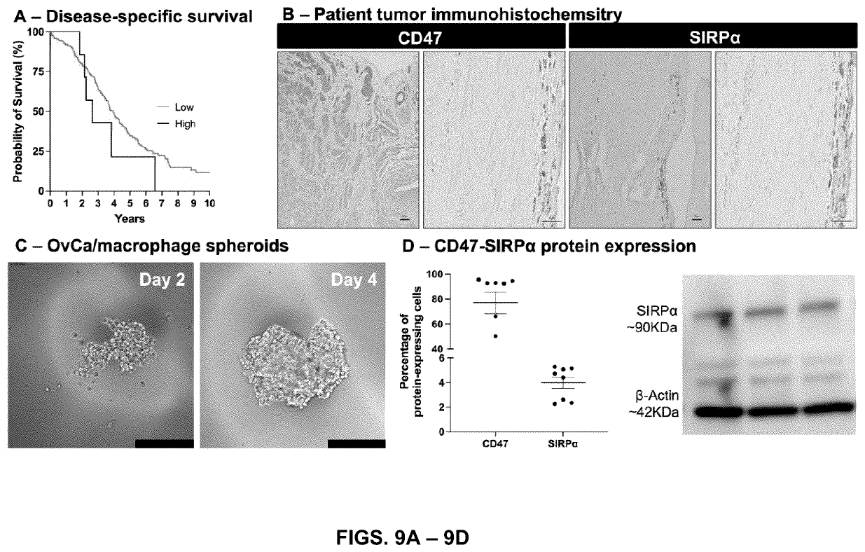

CD47-SIRPα axis is clinically significant in ovarian cancer. The CD47 and SIRPα proteins are negatively associated with OvCa progression, making them highly relevant targets for therapeutic discovery. Specifically, median disease specific survival is reduced by 1.5-fold (p=0.25) in cases of high (top 10%) SIRPα expression in OvCa patients (FIG. 9A). Primary patient tumors from a high grade serous OvCa patient express both CD47 and SIRPα, as evidenced by the presence of the brown immunohistochemistry stain (FIG. 9B), further highlighting the role of this macrophage immune checkpoint in the context of OvCa progression.

Considering the correlation between patient outcome and macrophage checkpoint signaling in clinical OvCa, a heterospheroid model was utilized to study these interactions in vitro. OVCAR3 cells were seeded on the hanging drop array with THP-1-derived M0 macrophages to create co-culture spheroids (FIG. 9C). The aggregation of OVCAR3 and M0 macrophages into a heterospheroid entity is visualized at days 2 and 4 (FIG. 9C). Following four days of spheroid formation, flow cytometry analysis showed that 77.05±8.76% of cells in heterospheroids expressed CD47 and 3.99±0.47% of cells expressed SIRPα (FIG. 9D, left). Similarly, SIRPα expression was also identified via western blotting, appearing as a band at 90 KDa (FIG. 9D, right). Together, these results indicated the maintenance of a SIRPα-expressing macrophage population within OvCa/macrophage heterospheroids.

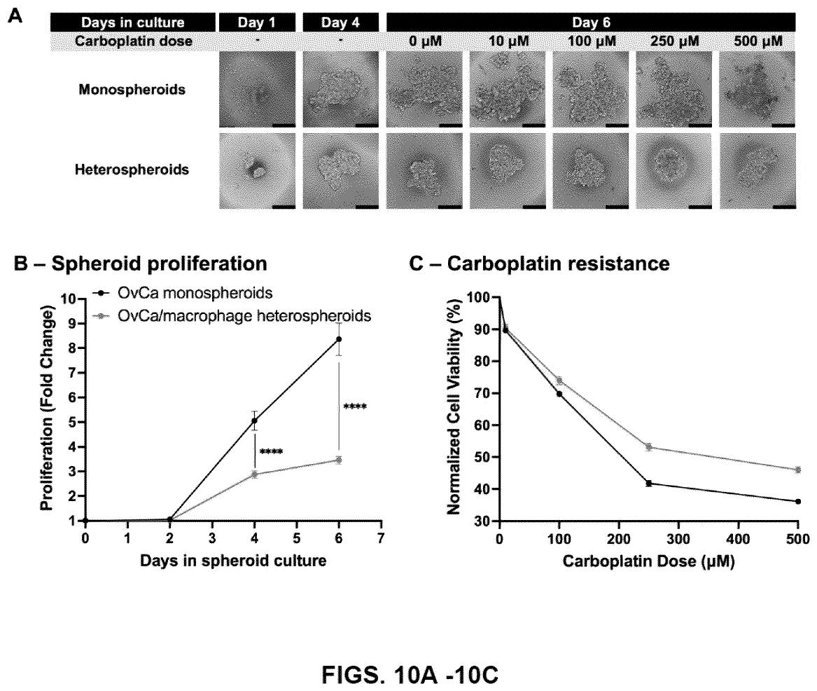

Macrophages impact OvCa growth and chemoresistance. Spheroids formed from 100 OvCa cells (monospheroids) or 50 OvCa with 50 macrophages (heterospheroids) were cultured for 6 days for analysis of proliferation over time. FIGS. 10A-10C demonstrate macrophage-dependent chemoresistance in 3D spheroids. FIG. 10A is a set of phase contrast micrographs of monospheroids (OvCa cells alone) and heterospheroids (OvCa/macrophage coculture). By day 4, a single compact spheroid could be seen with defined margins (FIG. 10A). On days 4 and 6, MTS viability measurements were compared to those taken at the time of spheroid initiation (day 0) to produce a fold change in proliferation over time. Despite seeding 100 cells/drop in both conditions (day 0), monospheroids proliferated faster than heterospheroids, showing 5.06±0.39-fold and 8.37±0.66-fold changes at days 4 and 6 compared to 2.87±0.16-fold and 3.46±0.16-fold in heterospheroids, respectively (****p<0.0001, two-way ANOVA, FIG. 10B). Visual confirmation of cell proliferation can be observed in phase contrast micrographs where mono-and heterospheroids are similarly compact in structure, but monospheroids appear larger in cell number (FIG. 10A). Compact spheroids were treated on day 4 with the platinum-based chemotherapy drug, carboplatin (0-500 μM). Following 48 hours of incubation with the drug, viability was determined using the MTS assay, from which IC₅₀ values for carboplatin were determined. Carboplatin treatment decreased cell viability in both OvCa monospheroids and OvCa/macrophage heterospheroids. This was visually apparent at Day 6 (2 days following carboplatin treatment), where monospheroids had less defined spheroid boundaries surrounded by increased amounts of cellular debris (FIG. 10A). FIG. 10B is a graphical representation of the proliferation measured by MTS viability assay confirmed the increased sizes observed in monospheroids compared to heterospheroids in spheroid images. Monospheroids (black trace) grew nearly 2.4 times more than heterospheroids (orange trace) by day 6 owing to the terminal differentiation state of macrophages which inhibited their proliferation. Shifts in viability curves were quantified. FIG. 10C is a graphical representation of the monospheroid and heterospheroid response to carboplatin chemotherapy (0-500 μM) was measured with an MTS viability assay. Heterospheroids were 1.69-fold more resistant to carboplatin with IC₅₀ value of 368.2±37.70μMas compared to 218.1±35.13 μM in monospheroids (FIG. 10C).

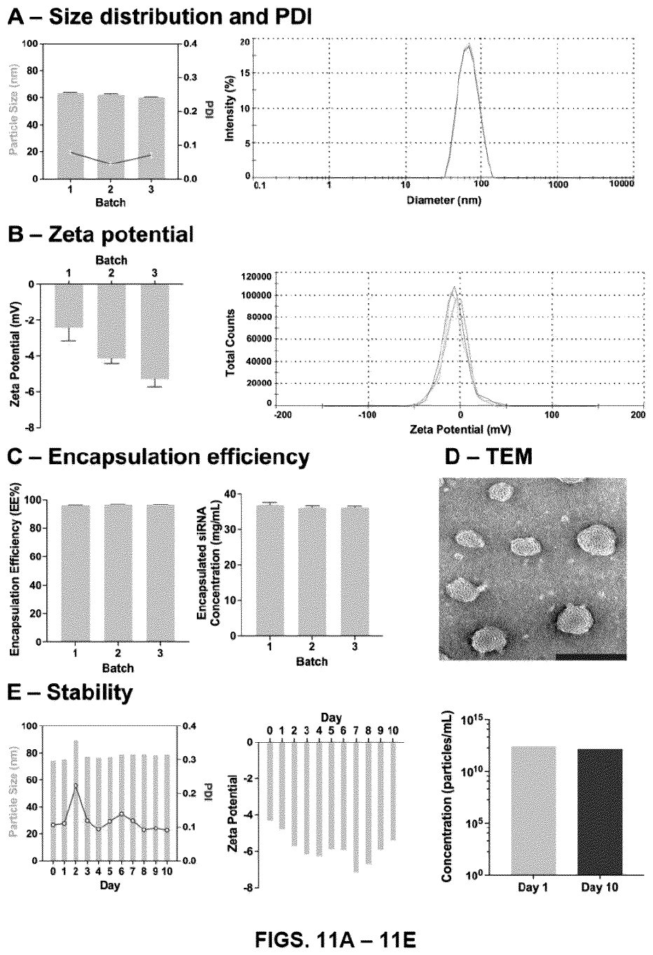

Macrophage checkpoint nano-immunotherapy design and characterization. Major obstacles of RNA therapies include nucleic acid stability and off-target effects or premature drug clearing. To improve transport and specificity of SIRPα siRNA (siSIRPα) as a macrophage-targeted immunotherapy, lipid nanoparticles (LNP) were designed to encapsulate and deliver the siRNA. siSIRPα LNP were evaluated for particle size, polydispersity index (PDI), zeta potential, encapsulation efficiency (EE) and RNA concentration. Reproducibility and stability were assessed for three separate batches prepared at least one week apart and tracked over 10 days. The particles were formulated with an average diameter of 62.32±0.56 nm (FIG. 11A). The LNP exhibited very uniform size distribution and PDI (<0.1, FIG. 11A). Zeta potential also remained consistent at −3.95±1.45 mV (FIG. 11B). The EE was >95% and the concentration of encapsulated siRNA was 36.41±0.39 μg/mL (FIG. 11C). LNP size, PDI, zeta potential, RNA contents and encapsulation were very reproducible with less than 10% variation between the batches for all measured criteria. These values also remained constant over 10 days, confirming the stability of the LNP over the course of the experiments (FIG. 11E). TEM of siSIRPα LNP confirmed the uniform size distribution as well as the bilayer structure of the delivery system (FIG. 11D).

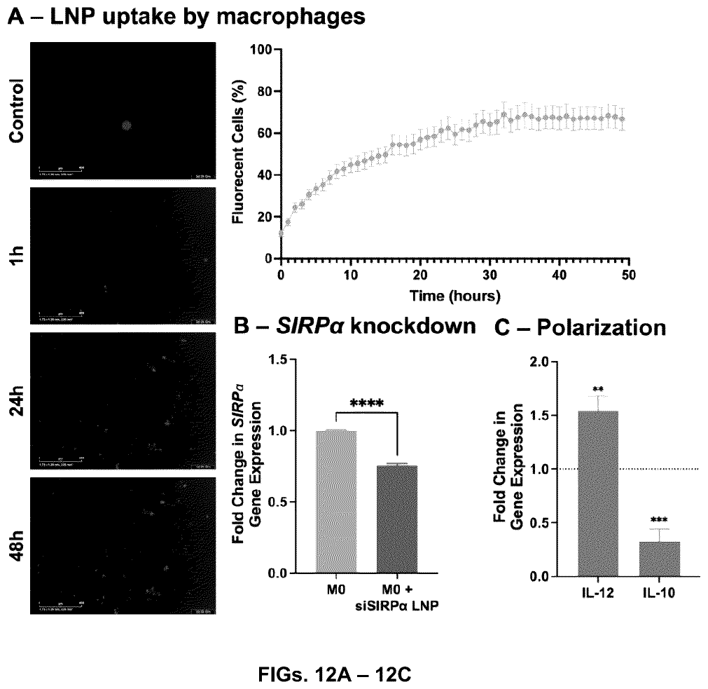

The siSIRPα LNP uptake and alteration of macrophage phenotype. Macrophage uptake of siSIRPα LNP reduced the gene expression of SIRPα. In vitro kinetic studies were conducted to test LNP uptake by macrophages, using naïve M0 monocyte-derived THP-1 macrophages over 48 hours. Macrophage uptake of red fluorescent LNP was evident by the increase in red fluorescence over 48 hours (FIG. 12A). Naïve M0 macrophages were able to efficiently engulf siSIRPα LNP as visualized in images tracking fluorescently labeled particles. As expected, macrophage engulfment of LNP started immediately upon their addition to macrophage cultures (1 hour, FIG. 12A). By 30 hours, more than 50% of cells showed red LNP-associated fluorescent signal indicating particle uptake (FIG. 12A, left). FIG. 12A on the right is a graphical representation of the percentage of fluorescent cells over time. Analysis of hourly images over 48 hours showed more than 50% of cells uptake the LNP by 30 hours (FIG. 12A, right). FIG. 12B is a graphical representation of the gene expression analysis indicating that macrophages treated with siSIRPα LNP expressed 24.28±1.34% less SIRPα than untreated controls. (****p<0.0001, unpaired t test). FIG. 12C is a graphical representation of the polarization of the M0 macrophages towards an M1 phenotype. Addition of siSIRPα LNP to M0 macrophages induced a shift toward an anti-tumoral (M1) phenotype defined by a 1.54±0.14-fold increase in IL-12 expression and a corresponding 0.33±0.12-fold decrease in IL-10 expression.

Following 24 hours of treatment, LNP caused a 24.28±1.34% knockdown in M0 macrophages differentiated from THP-1 monocytes (****p<0.0001, unpaired t test, FIG. 12B). The siSIRPα LNP treatment was evaluated for effects on naïve macrophage activation. Interestingly, knocking down SIRPα expression increased pro-inflammatory M1-like gene expression (IL-12; ˜1.5-fold, **p<0.00, two-way ANOVA, FIG. 12C) and decreased IL-10 (˜0.4-fold, ***p<0.001, two-way ANOVA, FIG. 12C) indicating the potential of siSIRPα LNP to influence M1-like programming in macrophages.

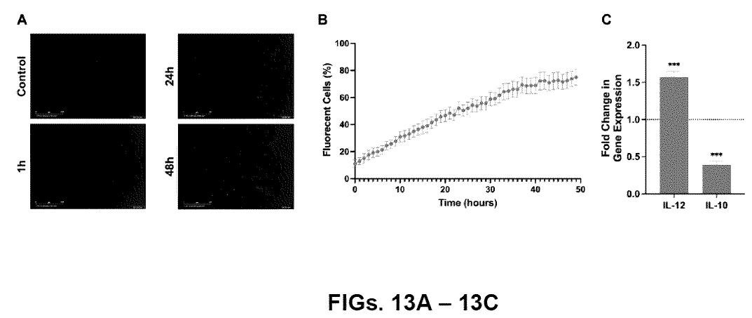

Even alternatively activated macrophages (M2-like) had robust uptake of siSIRPα LNP (FIGS. 13A-13C). FIGS. 13A-13C present the siSIRPα LNP uptake by M2 macrophages. The immunosuppressive nature of the OvCa tumor microenvironment can drive macrophage polarization toward alternative activation. As such, the ability of such a macrophage population to uptake siSIRPα LNP was tested and the subsequent effect of the LNP on macrophage phenotype was analyzed. FIG. 13A presents the live-cell imaging of THP-1-derived macrophages that were polarized to M2-like phenotypes with MCSF and IL-4. M2 macrophages efficiently took up fluorescently labeled siSIRPα LNP as indicated by red signal in live cell images taken over 48 hours after LNP administration. Uptake continued over 48 hours with increased red fluorescence visible with time. FIG. 13B is a graphical representation of the image quantification confirmed this uptake efficiency showing that >50% of cells exhibited fluorescence by 30 hours. FIG. 13C is a graphical representation of the polarization of the M2 macrophages towards an M1 phenotype. Following confirmation of particle uptake, M2 macrophages treated with siSIRPα LNP were evaluated for LNP ability to shift polarization. LNP did induce a more M1-like gene signature with a significant increase in IL-12 and a corresponding reduction in IL-10 M2 expression (***p<0.001). This indicated the ability to reverse the pro-tumoral activation state commonly expressed by macrophages in the OvCa tumor microenvironment.

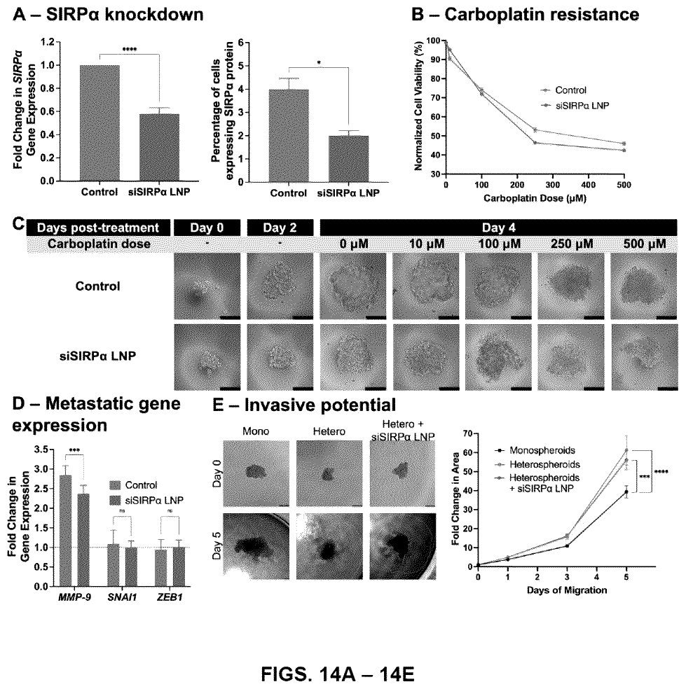

The siSIRPα LNP efficiency in OvCa macrophage heterospheroids. Heterospheroids formed from OVCAR3 cells and THP-1-derived macrophages were treated with 25 nM siSIRPα LNP. After 48 hours of incubation with the siSIRPα LNP, gene and protein expression were quantified to evaluate the nano-immunotherapy efficiency in reducing SIRPα expression in an OvCa/macrophage heterospheroid setting. LNP treatment produced a 41.97±5.25% knockdown in SIRPα gene expression compared to untreated controls (****p<0.0001, unpaired t test, FIG. 14A). Further characterization was provided by flow cytometry analysis which showed similar reduction of SIRPα expression in heterospheroids upon LNP treatment (compare 3.99±0.47% of untreated to 2.01±0.22% of LNP treated cells expressing the SIRPα surface protein, *p<0.02, unpaired t test, FIG. 14A). FIGS. 14A and 14B are graphical representations of the effects of siSIRPα LNP treatment of heterospheroids. This treatment resulted in a reduction of nearly 42% (****p<0.0001) of SIRPα gene expression (FIG. 14A, left) which translated to a similar 50% reduction in protein expression (FIG. 14A, right). This decrease in SIRPα expression was linked to a recovery of chemotherapy response in resistant heterospheroids (FIG. 14B). In fact, siSIRPα LNP induced a 32% recovery of the resistance caused by macrophages as compared to OvCa monospheroid controls. FIG. 14C is a set of phase contrast micrographs of control and LNP-treated heterospheroids. Images showed slowed growth by day 2 following LNP treatment. LNP-treated heterospheroids were more sensitive to carboplatin chemotherapy, showing increased cell death by day 4 across 5 doses used to generate an IC₅₀ value. FIG. 14D is a graphical representation of the metastasis-indicating gene analysis of OvCa cells. Cells from heterospheroids were treated with siSIRPα LNP or maintained as controls and sorted to analyze OvCa cells alone. Both heterospheroid conditions were compared to macrophage-naïve OVCAR3 cells from monospheroids. Loss of MMP-9 gene expression was observed after siSIRPα LNP (compare 2.37-fold 2.85-fold increase in MMP-9 in control heterospheroids to 2.37-fold after LNP, ***p=0.0005, two-way ANOVA) which indicated OvCa cells becoming less invasive following LNP treatment. Similarly, there is a small decrease in epithelial-to-mesenchymal transition gene SNAI1. FIG. 14E on the left is a set of photographs showing the spheroid migration model created to measure OvCa invasiveness. Spheroids were maintained in hanging drop culture for 4 days with siSIRPα LNP treatment on day 2. After seeding onto 2D well-plates, cellular migration was tracked via changes in area occupied by cells as they traveled away from the spheroid as indicated by the colored outlines drawn in ImageJ software (left). FIG. 14E, right panel, is a graphical representation of the migratory ability. Macrophage presence induced a significant increase in migratory ability (****p<0.0001, two way ANOVA) which was partially reversed in spheroids which were pre-treated with LNP prior to seeding (right).

The siSIRPα LNP manipulate OvCa metastatic behavior. After confirming the knockdown efficiency of the siSIRPα LNP immunotherapy, functional repercussions of interfering with “Don't Eat Me” signaling between OvCa cells and macrophages within heterospheroids was tested by quantifying changes in OvCa response to carboplatin chemotherapy as well as metastasis-indicating gene expression and invasive potential.

The siSIRPα LNP treatment increased heterospheroid sensitivity to carboplatin, first visually apparent by the loss of heterospheroid integrity in carboplatin treated conditions that also received LNP treatment (FIG. 14C). Quantification of cell viability subsequently demonstrated that siSIRPα LNP treatment trended towards reversed carboplatin resistance in OvCa/macrophage heterospheroids (compare improved IC₅₀=320.7±54.57 μM compared to no siSIRPα treatment IC₅₀=368.2±37.70 μM; FIG. 14B).

Importantly, siSIRPα LNP treatment did not impact OvCa monospheroid viability or chemosensitivity to carboplatin. Heterospheroids treated with negative control (scramble) siRNA NP also maintained stable viability and IC₅₀ indicating there is no negative effects of the vehicle itself. Taken together, these data suggest the targeted effect of siSIRPα LNP on macrophages within OvCa/macrophage heterospheroids, impacting platinum chemotherapy sensitivity.

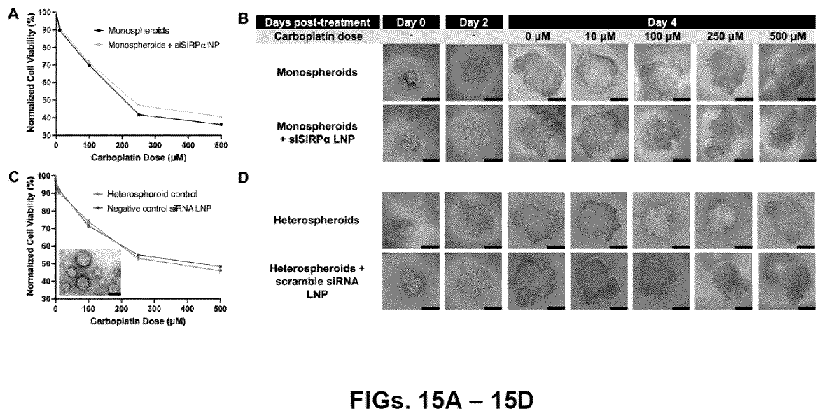

FIGS. 15A-15D present the carboplatin sensitivity in control spheroids. siSIRPα LNP treatment was shown to slow growth and decrease viability in chemotherapy-treated heterospheroids. To confirm whether this was a toxic effect of the LNP or rather OvCa growth reduction resulting from CD47-SIRPα inhibition, additional controls were tested in the presence of LNP. FIG. 15A is a graphical representation of the cell viability in the OvCa monospheroids in response to carboplatin with and without siSIRPα LNP treatment. OvCa monospheroids were similarly responsive to carboplatin with and without siSIRPα LNP treatment (compare IC₅₀ value of 218.1 μM in control spheroids to 272.1 μM after siSIRPα LNP). As hypothesized, LNP did not appear to have a toxic effect on the OvCa cells as evidenced by this maintenance of cell viability. FIG. 15B is a set of associated phase contrast micrographs that show comparable spheroid size and morphology after 2 days of LNP treatment and after another 2 days with 0-500 μM chemotherapy confirming the LNP are not acting on OvCa cells to reduce viability. FIG. 15C is a graphical representation of the cell viability in the OvCa monospheroids in response to carboplatin with and without control LNP containing negative control (scramble) siRNA. These LNPs were administered to OvCa/macrophage heterospheroids to evaluate the potential adverse effects of the LNP vehicle on spheroid viability. A transmission electron micrograph (50,000×; scale bar=50 nm) illustrates the uniform size distribution of negative control LNP (insert in FIG. 15C). FIG. 15D is a set of associated phase contrast micrographs. Heterospheroids did not exhibit any signs of toxicity in response to the negative control LNP as evidenced by similar IC₅₀ values (368.2 μM with no treatment and 405.3 μM with scramble LNP) and phase contrast micrographs (scale bar=200 μm).

OvCa cells were separated from macrophages following co-culture in heterospheroids by GFP-based fluorescence associated cell sorting. The presence of macrophages in OvCa spheroids increased MMP-9 signaling indicative of OvCa metastatic potential. This macrophage effect was reduced by blocking CD47-SIRPα signaling with siSIRPα LNP treatment (***p<0.001, two-way ANOVA, FIG. 14D). The SNAI1 gene was also slightly reduced following siSIRPα LNP treatment.

More than 80% of OvCa is already metastatic at the time of diagnosis. Metastatic OvCa is more invasive and resistant to chemotherapy than localized disease giving rise to survival rates of grave concern. Once OvCa cells exfoliate from the primary tumor, they aggregate within the malignant ascites, where they interact with many other stromal cells including macrophages. OvCa/macrophage interactions drive carboplatin chemoresistance and invasiveness, contributing to malignant progression. In the context of OvCa/macrophage interactions, the macrophage checkpoint CD47-SIRPα has been identified as the target for the compositions and methods disclosed herein. Overexpression of OvCa CD47 has been linked to metastasis and devastating prognosis. Not only do patient samples of advanced OvCa demonstrate positivity for CD47-SIRPα staining (FIG. 9A), heterospheroids bioengineered from the cell line OVCAR3 combined with THP-1 macrophages also robustly express CD47-SIRPα (FIG. 9D).

In the OvCa/macrophage CD47/SIRPα-expressing heterospheroid model, reciprocal interactions between OvCa cells and macrophages drive carboplatin chemoresistance (FIGS. 10A-10C). Heterospheroids generated by co-culturing OvCa cells and macrophages were 2.5 times less proliferative than OvCa cells alone (FIG. 10B). Macrophages, being the terminal result of monocytic differentiation, lose their ability to proliferate which would account for this reduction. The presence of macrophages decreased OvCa sensitivity to platinum chemotherapy by more than 1.5 times, indicating their contribution to a chemoresistant cancer phenotype (FIG. 10C). This finding connects macrophages, via a variety of signaling pathways, to OvCa chemoresistance and ultimately metastasis.

CD47 binding to macrophage SIRPα contributes to OvCa progression, making it an attractive therapeutic axis to target. In fact, the first in-human, first-in-class CD47 antibody had positive partial results in prolonging survival of patients with advanced ovarian cancer. However, anti-CD47 antibodies have historically been studied for disrupting CD47-SIRPα signaling. These therapies have met obstacles due to a lack of treatment specificity and the resulting adverse effects.

Disclosed here are composition containing short interfering RNA (siRNA) that knock down the expression of macrophage SIRPα of the CD47-SIRPα axis as part of liposomes and other lipid nanoparticles (LNP). LNP are formed by the self-assembly of RNA, phospholipids and cholesterol, with case of manufacture and tight quality control (FIGS. 11A-11E). An additional benefit of nanotherapy for this precise application leverages the intrinsic phagocytic property of macrophages. Nanoparticles, as circulating solid particles foreign to the body, have a high efficiency for targeting macrophages due to the cells' key role in uptake, processing and clearing of such substances. Therefore, the compositions described herein take advantage of the natural affinity of LNP to macrophages to direct an siRNA-based knockdown of macrophage SIRPα. The SIRPα siRNA (siSIRPα) LNP had a diameter around 60 nm with zeta potential measuring approximately −4 mV (FIGS. 11A-11B). These properties are desirable based on a variety of studies indicating maximum cellular uptake of particles 40-60 nm in diameter. Additionally, a neutral zeta potential (range −10 to 10 mV) can lead to better stability and increased half-life of particles in circulation, creating a higher chance of intact delivery to the target tissue. The particles efficiently and consistently encapsulated siSIRPα while maintaining uniform particle characteristics over experimental timelines (FIGS. 11A-11E).

The uptake of fluorescently labeled siSIRPα LNP was evident (FIG. 12A) regardless of alternatively activated macrophage activation status (FIGS. 13A-13C) which might typically be observed in the OvCa tumor microenvironment. In fact, uptake of siSIRPα LNP led to active macrophage re-polarization (of both naïve and alternatively activated macrophages), evident by increases in gene expression of IL-12 with a concomitant decrease in gene expression of IL-10 (FIG. 12C, FIG. 13C). Such macrophage reprogramming has been reported previously with the disruption of the CD47-SIRPα macrophage immune checkpoint axis.

In order to evaluate the siSIRPα LNP therapeutics, the in vitro 3D bioengineered model of OvCa/macrophage heterospheroids was utilized, focusing on malignant progression readouts like carboplatin chemoresistance and invasiveness. siSIRPα LNP treatment reduced the expression of SIRPα in OvCa/macrophage heterospheroids by ˜50% (FIG. 14A). As expected, siSIRPα LNP treatment led to increased sensitivity (lowered chemoresistance) to carboplatin treatment (FIG. 14B) likely due to macrophage reprogramming. Disrupting CD47-SIRPα signaling further influenced OvCa metastatic phenotype by reducing metastasis-indicating MMP-9 gene expression and cellular invasive potential in this system (FIG. 14D-FIG. 14E). These data indicate the relevance of specifically manipulating this macrophage-induced OvCa metastasis.

In Vivo Efficacy Assessment of siSIRPα LNP. Animal protocols were approved by the Institutional animal care and use committee (IACUC) at the Houston Methodist Research Institute. All animal experiments were conducted in accordance with approved protocols. Female C57BL/6 mice of at least 10 weeks of age were purchased from Charles Rivers Laboratories. The mice were housed with controlled temperature (25° C.), 12:12 hour lighting cycle, and access to standard diet and water. Mice received intraperitoneal injections with 5×10⁶ ID8 Luc expressing murine ovarian cancer (OvCa) cells (in 200 μL) (D0). The tumor growth was followed for 21 days prior to starting the therapy. On D21, mice were imaged for bioluminescence signal on IVIS System (Perkin Elmer, Waltham, MA) for ID8 OvCa growth. Mice were then treated with three rounds of therapy as described below in “groups” every 3 days (9 days in total). After each therapy the animals were injected with luciferin and imaged with IVIS to record the tumor burden based on luminescence. The treatment groups were: Group 1: siSIRPα LNP (5 μg in 50 μL, IV)+Carboplatin (1.0 mg/kg, IV); Group 2: PBS (50 μL, IV)+Carboplatin (1.0 mg/kg, IV); Group 3: PBS (50 μL, IV)+PBS (50 μL, IV). IVIS imaging was done for whole abdomen bioluminescence expression. Animals were sacrificed after the final IVIS imaging (D30). Organs and blood serum were harvested from animals, imaged by IVIS and processed for endpoint analyses (IHC staining for Ki67, M1/M2 macrophages; SIRPα knockdown; cytokine profile).

Immunohistochemistry (IHC) Staining. Paraffin embedded mice organs were section cut for IHC staining. Sections were treated with citrate buffer (ThermoFisher, Waltham, MA) for antigen retrieval and then blocked with 1% hydrogen peroxide (H2O2) (ThermoFisher, Waltham, MA) in 1% bovine serum albumin (BSA) (Sigma-Aldrich, St. Louis, MO). Sections were then reacted with primary antibody (PA5-19462, ThermoFisher, Waltham, MA) at 40C overnight and then secondary antibody (goat anti-rabbit IgG H&L HRP #ab408443, Abcam, Cambridge, UK) for 2 hr at room temperature. Staining was visualized by DAB (SK4100, Vector, Newark, CA) and counterstained by Harris Hematoxylin (ThermoFisher, Waltham, MA). Processing of positive staining was done through Olympus cellSens software (Olympus, Hachioji, Japan) and statistically analyzed through GraphPad Prism 10.

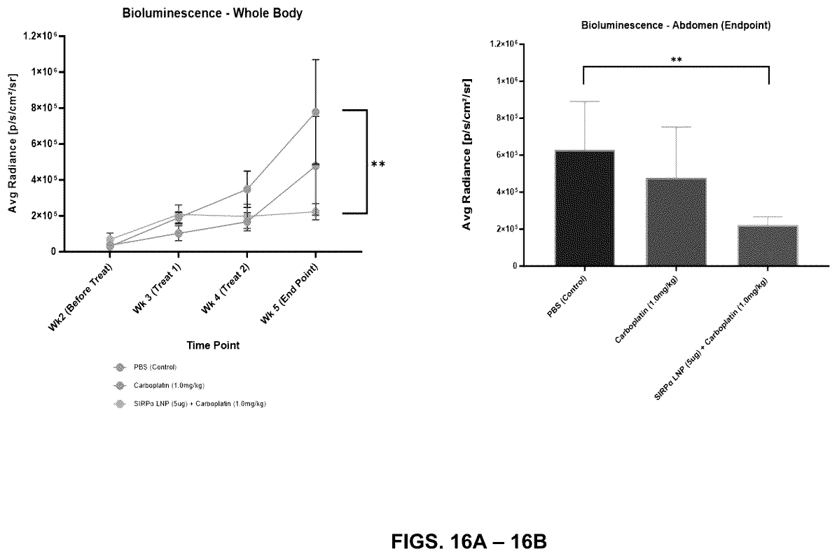

ID8/C57 IVIS Luminescence Imaging. Bioluminescence assessment of ID8 Luc OvCa carrying mice (both whole body and abdomen at the end point) showed significant decrease of luciferase ID8 OvCa signal in the SIRPα siRNA (5 μg)+Carboplatin (1.0 mg/kg) treatment group when compared to 1×PBS treatment group over study time course (week 2-week 5) in mice whole body and abdomen focused scans. FIGS. 16A-16B are graphical representations of the ID8/C57 IVIS luminescence imaging of both the whole body (FIG. 16A) and abdomen (FIG. 16B) at the end point. The ID8/C57 IVIS luminescence imaging shows decreased tumor burden in the LNP carrying SIRPα siRNA (5 μg)+Carboplatin (1.0 mg/kg) group, as the cells are tagged with luminescent luciferase.

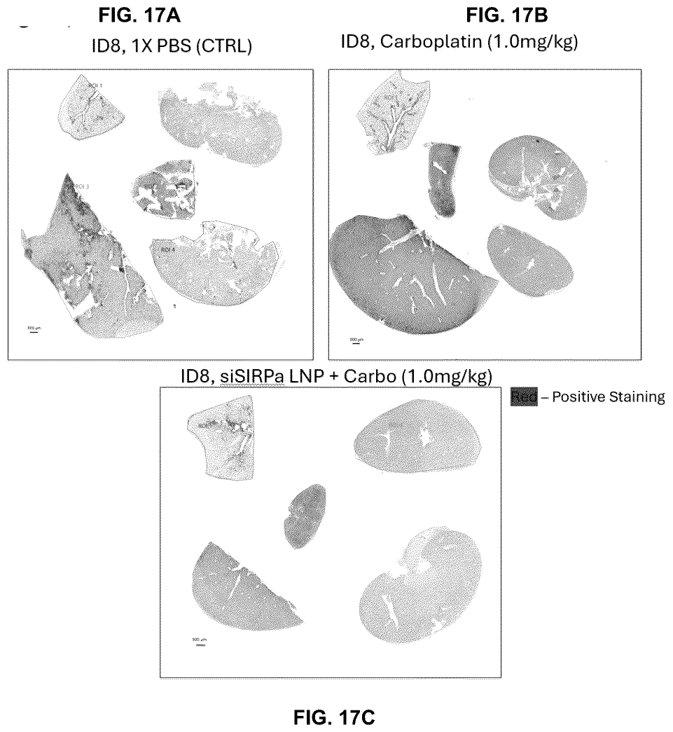

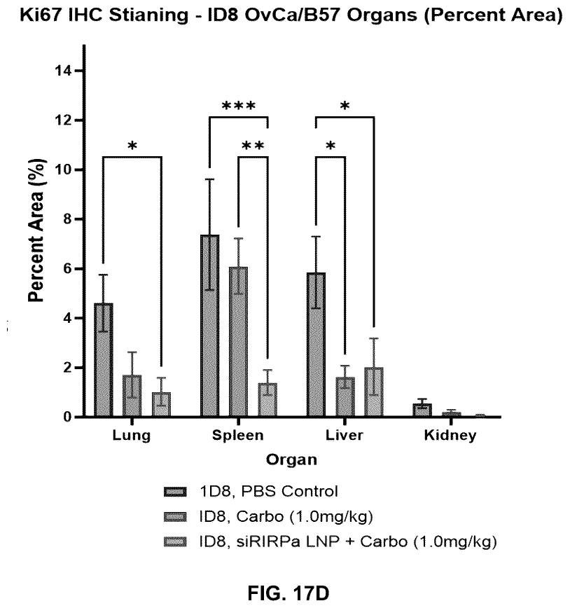

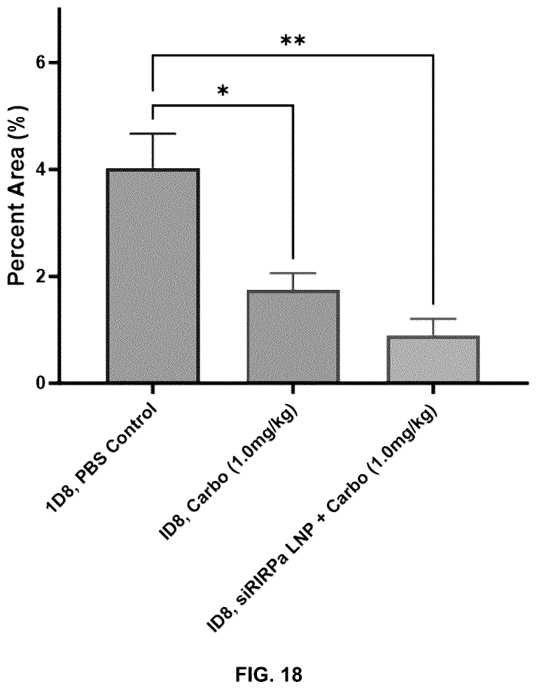

Ki67 Staining. Ki67 immunohistochemistry (IHC) staining of mice organs exhibited significant decrease of Ki67 expression (protein marker of cell proliferation, ThermoFisher, PA5-19462) in individual lung, spleen, liver, and kidney tissue and total aggregate expression in organs of mice treated with SIRPα siRNA (5 μg)+Carboplatin (1.0 mg/kg) when compared to mice treated with 1×PBS. FIGS. 17A-17C present Ki67 staining in individual lung, spleen, liver, and kidney tissue of mice treated with SIRPα siRNA (5 μg)+Carboplatin (1.0 mg/kg) when compared to mice treated with 1×PBS. FIG. 17D is a graphical representation of the Ki67 staining in the total aggregate expression in organs of mice treated with SIRPα siRNA (5 μg)+Carboplatin (1.0 mg/kg) when compared to mice treated with 1×PBS. Decreased expression of Ki67, indicative of decreased ID8 OvCa tumor cell proliferation and burden, was observed in siSIRPα LNP (5 μg)+Carboplatin (1.0 mg/kg) group in individual abdominal organs and in the all organs. FIG. 18 is a graphical representation of the Ki67 IHC staining analysis of all organs that present significant lowering of the proliferation marker with treatment group siSIRPα LNP (5 μg)+carboplatin (1.0 mg/kg).

OvCa CD47 signaling to macrophage SIRPα directly inhibits the anticancer response of macrophages. This permits OvCa survival in the presence of phagocytic macrophages. However, the data provided herein indicate not only survival, but increased OvCa metastatic signatures as a result of OvCa/macrophage interactions. Compositions disclosed herein disrupt this trophic axis, to improve patient outcomes in advanced metastatic ovarian cancer.

The hanging drop co-culture spheroids uniquely models the ascites transport of metastatic tumor clusters and permits the isolation and study of the relevant cellular interactions. This platform has been leveraged to develop a novel therapeutic strategy targeting macrophages with lipid nanoparticles specifically designed to safely deliver siRNA to phagocytic macrophages in the tumor microenvironment and reduce SIRPα expression. These compositions and methods provide improved therapeutics targeting metastatic OvCa in the clinic to improve therapy response and ultimately patient survival.

### MATERIALS AND METHODS

Materials. Cell culture reagents and all materials used for western blotting were purchased from ThermoFisher Scientific (Waltham, MA) unless otherwise specified. Roswell Park Memorial Institute (RPMI) 1640 Medium (Gibco, Grand Island, NY) supplemented with 10% heat-inactivated fetal bovine serum (FBS; Peak Serum, Inc., Wellington CO) and 1× Antibiotic-Antimycotic solution was used as growth medium. Lentivirus encoding the green fluorescent protein (GFP) was obtained from the University of Michigan Vector Core to transduce OvCa cells. 1,2-distearoyl-sn-glycero-3-phosphocholine (DPSC) and 1,2-distearoyl-sn-glycero-3-phosphoethanolamine-N-[methoxy (polyethylene glycol)-2000](ammonium salt) (DSPE-PEG (2000)) were purchased from Avanti Polar Lipids (Alabaster, AL), cholesterol from Sigma-Aldrich (St. Louis, MO), 4-(dimethylamino)-butanoic acid, (10Z,13Z)-1-(9Z,12Z)-9,12-octadecadien-1-yl-10,13-nonadecadien-1-yl ester (MC3) from MedChemExpress (Monmouth Junction, NJ) and citrate buffer from ThermoFisher Scientific.

SIRPα-dependent patient survival. Survival analysis was conducted using data publicly available on the University of California-Santa Cruz Xena Browser. The TCGA Ovarian Cancer dataset (TCGA OV) was analyzed to categorize patients into high and low SIRPα expression where expression levels in the top 10% were considered high (9 of 278 patients). Categorized disease-specific survival data was exported and plotted using the Kaplan-Meier method in GraphPad Prism.

Immunohistochemistry of patient sample for SIRPα and CD47 expression. Primary patient tumor tissues were collected from consenting patients (IRB #PRO00029517, MOD00005725, Houston Methodist Hospital). Pathology evaluation demonstrated histologic findings consistent with high-grade serous OvCa. Tissue processing, sectioning and staining were performed at the Houston Methodist Pathology Core. Paraffin-embedded tumor samples were sectioned and deparaffinized using xylene and ethanol washes. Heat-mediated antigen retrieval was completed with Tris-EDTA (pH=9.0). Slides were incubated with hydrogen peroxide to eliminate endogenous peroxidase activity, and horse serum blocking buffer to reduce nonspecific binding of antibodies. Primary monoclonal antibodies for CD47 (1:100) or SIRPα (1:250; Novus Biologicals, Littleton, CO) were incubated with samples overnight at 4° C. followed by a 30-minute incubation with secondary antibodies at room temperature. DAB Substrate-Chromogen was allowed to incubate until the appropriate color change was visualized as compared to positive control samples. Slides were counterstained with Hematoxylin. A BZ8000 microscope (Keyence, Osaka, Japan) was used to acquire brightfield images of samples at 20× and 40× magnification.

Cell culture. The high-grade serous OvCa cell line, OVCAR3, and THP-1 monocytes were purchased from American Type Culture Collection (ATCC; Manassas, VA). They were maintained in complete RPMI culture medium at 37° C. with 5% CO₂. OvCa cells were transduced with lentiviral GFP following previously established protocols. Cells were grown in traditional 2D culture until seeding into 3D spheroids (see Methods 2.5). Monocytes were differentiated into M0 macrophages using 1 ng/mL phorbol-12-myristate-13-acetate (PMA; Sigma-Aldrich) for 72 hours on hanging drop spheroid arrays.

Spheroid formation on 384-well hanging drop arrays. OvCa monospheroids or OvCa/macrophage heterospheroids were formed on a 384-well hanging drop array following well-established protocols. OvCa monospheroids were initiated with 100 cells in a 20 μL drop of growth medium. To create OvCa/macrophage heterospheroids, the hanging drop platform was first leveraged to differentiate THP-1 monocytes to M0 macrophages by phorbol ester exposure20. Following a 3-day differentiation, macrophages were harvested and mixed with OvCa cells (50:50) to create heterospheroids. Monospheroid or heterospheroid formation was visually confirmed using phase contrast microscopy at day 4.

Flow cytometry. OvCa/macrophage heterospheroids were harvested for flow cytometry on day 4. Spheroids were mechanically dissociated into single cell suspensions in PBS supplemented with 2% FBS. Cells were incubated with CD47 or SIRPα antibody tagged with AlexaFluor 647 (BD Biosciences, Haryana, India) for 30 minutes at 37° C. Isotype controls were used to establish background staining for AlexaFluor 647. After washing away unbound antibody and resuspending in fresh buffer, flow cytometry analysis was performed using the Attune N×T (ThermoFisher Scientific). Unstained and isotype controls were employed to establish a gating strategy, cutting off a background gate at 0.5%. The percentages of cells expressing CD47 or SIRPα were determined in OvCa/macrophage heterospheroids.

Western blot SIRPα protein identification. Protein was extracted from heterospheroids with RIPA buffer and quantified using the Pierce BCA Protein Assay Kit. 20 μg protein was loaded into each lane of a Novex trisglycine mini protein gel (4-20%) and transferred to a low-fluorescence PVDF transfer membrane along with the PageRuler Prestained Protein Ladder (10-180 kDA). The membrane was blotted with a SIRPα polyclonal antibody as well as β-Actin for a loading control. A horseradish peroxidase (HRP)-conjugated secondary antibody (rabbit antihuman IgG; Invitrogen) was used to visualize bands on the LI-COR C-Digit 3600 Western Blot Scanner.

MTS cell proliferation assay. An MTS assay (Abcam, Cambridge, UK) was used to assess cell proliferation over time. Spheroids were evaluated at days 0, 2, 4 and 6 to generate paired samples. At the timepoint of interest, cells were incubated with the MTS reagent diluted 1:10 in the 20 βL of culture medium of the hanging drop plate at 37° C. After a 2-hour incubation, absorbance was measured at 490 nm with the Cytation 7 microplate reader (BioTek, Winooski, VT). Reported values were normalized to a day 0 absorbance measurement for time-dependent proliferation analysis.

Response to chemotherapy. Carboplatin (MedChemExpress, Monmouth Junction, NJ) was dissolved by sonication in sterile PBS at a stock concentration of 5 mg/mL. OVCAR3 monospheroids or OVCAR3/macrophage heterospheroids formed over 4 days were treated with 0-500 μM carboplatin and incubated for 2 days/48 hours. An MTS assay was then used to determine the percent cell viability in carboplatin-treated spheroids compared to untreated controls. IC50 values were calculated with the GraphPad Prism normalized log (inhibitor) variable slope nonlinear fit function.

SIRPα siRNA (siSIRPα) LNP design. siSIRPα LNP were formulated using the Nanoassemblr Benchtop (Precision NanoSystems, Vancouver, BC, Canada) by mixing the ethanolic phase containing DPSC: DSPE-PEG2000: cholesterol: MC3 at the molar ratio 8:1.5:38.5:52 with the aqueous phase containing 50 μg/mL siSIRPα in 0.1 M citrate buffer (pH 5). One volume of ethanolic phase was mixed with three volumes of aqueous phase at a combined flow rate of 12 mL/min. Residual ethanol and non-encapsulated siRNA were removed from the solution with two dialyses in PBS (>4 hours each) using a D-Tube Dialyzer Maxi (12-14 kDa, Sigma-Aldrich). The systems were diluted in PBS to a final 500 nM working concentration.

In certain embodiments, the SIRPα siRNA LNP were prepared using a microfluidic device (Nanoassemblr Benchtop, Precision NanoSystems, Vancouver, BC, Canada) to mix one volume of the ethanolic phase with three volumes of the aqueous phase (Table 1). A dual syringe pump (Model S200, KD Scientific, Holliston, MA) was used to drive the solutions through the micro-mixer at a combined flow rate of 12 mL/min. Residual ethanol and non-encapsulated siRNA were removed from the solution with D-Tube Dialyzer Maxi (12-14 kDa, Sigma-Aldrich, St. Louis, MO) in phosphate buffered saline (PBS). Two dialysis washes of >4 hours were performed. The systems were diluted in PBS to a final working concentration of 500 nM.

TABLE 1
List of components of the ethanolic and aqueous phases used to fabricate SIRPα siRNA LNP.

| Ethanolic Phase | | | Aqueous Phase | | |
|---|---|---|---|---|---|
| Component | Amount | Source | Component | Amount | Source |
| 1,2-distenroyl-sn-glycero-3- | 1.05 μmole | Avanti Polar Lipids, | 0.1M Citrate Buffer | 800 μL | ThermoFisher |
| phosphochohne (DPSC) | | Alabaster, AL | pH 5 | | Scientific, |
| 1,2-distenroyl-sn-glycero-3- | 0.06 μmole | Avanti Polar Lipids, | | | Waltham, MA |
| phosphoethanolamine-N- | | Alabaster, AL | | | |
| [methoxy(polyethylene glycol)-2000] | | | | | |
| (ammoniumn salt) (DSPE-PEG(2000)) | | | | | |
| Cholesterol | 9.96 μmole | Sigma-Aldrich, St. | SIRPα siRNA | 50 μg/mL | Sigma-Aldrich. |
| | | Loms, MO | SaSI_Hs01_00017994 | | St. Louis, MO |
| D-Lin-MC3-DMA | 8.10 μmole | MedChem Express, | | | |
| | | Monmouth Junction, NJ | | | |

siSIRPα LNP characterization. The resulting siSIRPα LNP were characterized for particle diameter, polydispersity index (PDI) and zeta potential by dynamic light scattering (DLS; Malvern Zetasizer, Malvern Instruments, Malvern, UK) and for RNA content using the RiboGreen RNA Assay (ThermoFisher Scientific) according to the manufacturer's instructions. Encapsulated and non-encapsulated RNA were quantified. LNP were digested with 1% Triton X-100 (Sigma-Aldrich) to release RNA contents for quantification of total (encapsulated+unencapsulated) RNA. The encapsulation efficiency (EE) was calculated based on the formula:

$$EE = \frac{Encapsulated siRNA}{( Encapsulated + Unencapsulated siRNA )} \times 100 \%$$

For reproducibility, three different batches were prepared and tested. LNP were stored at 4° C. for up to 10 days after preparation at the final working concentration of 500 nM. Stability (size, PDI, zeta potential) was assessed daily over those 10 days. LNP Tracking Analysis (NTA) was utilized to quantify particle concentration at days 1 and 10 using the NanoSight NS300 (Malvern Panalytical, Malvern, UK). Particle samples were diluted (1:10,000) in nuclease-free water and measured for 30 seconds with manual shutter and gain adjustments on the 532 nm (green) laser. Three measurements were taken for each of the 3 samples and analyzed with NTA 3.4 Build 3.4.4 software. To confirm the LNP ultrastructure, Transmission Electron Microscopy (TEM) was performed in the Texas Heart Institute CV Pathology Core Electron Microscopy Laboratory. siRNA LNP (8-10 μL) were applied on a formvar/carbon-film coated mesh grid. Excess solution was blotted with filter paper and the samples allowed to air dry for 10 minutes at room temperature. Uranyl Acetate solution (0.2%, Sigma-Aldrich) was used as a negative stain. The samples were imaged under a JEOL 1230 transmission electron microscope (JOEL, Ltd, Tokyo, Japan) at 2-5×10⁴× magnification.

LNP uptake by macrophages. LNP uptake was assessed in naïve M0 macrophages. THP-1 monocytes were seeded at 4,000 cells/well in 96-well culture plates with 1 ng/ml PMA for 3 days. To monitor the uptake capabilities of macrophages, siSIRPα LNP were fluorescently tagged with 1,1′-Dioctadecyl-3,3,3′,3′-Tetramethylindocarbocyanine Perchlorate (DiL; ThermoFisher, Scientific). 25 nM siSIRPα LNP were added, and cells were maintained for 48 hours with hourly imaging on the Incucyte Live Cell Analysis system. Red fluorescence and phase images (10×, 5 images/well) were taken at each timepoint and analyzed using the Incucyte Live Cell Image Analysis software for average relative fluorescence units, per well, over time.

RNA extraction and qPCR. RNA was extracted from adherent macrophages or harvested heterospheroids with the RNeasy Mini Kit (Qiagen, Hilden, Germany). For gene expression analysis of invasiveness, a fluorescent activated cell sorting step was added to separate OVCAR3 cells cultured in heterospheroids with macrophages. The GFP tag on the OVCAR3 cells was used to separate the OvCa cells from macrophages on either a Beckman Coulter Moflo Astrios (Beckman Coulter, Brea, CA) or BDFACS Aria II (BD Biosciences) Cell Sorter at the Veterinary Pathobiology Flow Cytometry Facility and School of Medicine Analytical Cytometry Core, respectively, at Texas A&M University. RNA concentration and purity were measured with a NanoDrop OneC (ThermoFisher Scientific). RNA was converted to cDNA with the Applied Biosystems High Capacity cDNA Reverse Transcription Kit. qPCR was then performed using SYBR Green PCR MasterMix (ThermoFisher Scientific) on a QuantStudio 3 Real-Time PCR System (Applied Biosystems). Gene expression changes were calculated using the 2AACt method with GAPDH as a housekeeping gene. Gene primer sequences are listed in Table 2.

TABLE 2
Primer sequences for genes analyzed with qPCR

| | Forward primer | Reverse primer |
|---|---|---|
| Gene | sequence | sequence |
| GAPDH | CTGGGCTACACTGAGCACC | AAGTGGTCGTTGAGGGCAATG |
| SIRPα | GGCCTCAACCGTTACAGAGAA | GTTCCGTTCATTAGATCCAGT |
| | | GT |
| MMP-9 | TGTACCGCTATGGTTACACTCG | GGCAGGGACAGTTGCTTCT |
| SNAI1 | TCGGAAGCCTAACTACAGCGA | AGATGAGCATTGGCAGCGAG |
| ZEB1 | GATGATGAATGCGAGTCAGATG | ACAGCAGTGTCTTGTTGTTGT |
| | C | |
| IL-12 | CCTTGCACTTCTGAAGAGATTG | ACAGGGCCATCATAAAAGAGG |
| | A | T |
| IL-10 | GACTTTAAGGGTTACCTGGGTT | TCACATGCGCCTTGATGTCTG |
| | G | |

siSIRPα LNP immune checkpoint treatment. Mono-or hetero-spheroids were treated with 25 nM siSIRPα LNP on day 2 in hanging drop culture. LNP ability to reduce both macrophage SIRPα expression and OvCa metastatic behavior were evaluated over 4 more days.

Macrophage-mediated OvCa invasive potential. Monospheroids or heterospheroids treated with LNP or maintained as controls were seeded into 96-well culture plates following 4 days in hanging drop culture. Stacked phase contrast micrographs were taken of each spheroid over 5 days to monitor cell migration as a measure of invasiveness. The StackFocuser plugin on NIH ImageJ software was used to compress each image stack into a single focused 2D plane. Spheroid perimeters were drawn by hand in ImageJ to measure spheroid area across various timepoints. Fold change in area was calculated based on day 0 images of spheroids immediately after transfer from hanging drop culture. Increased spheroid area was used to track cell migration over time indicating OvCa invasive potential.

Statistical analysis. GraphPad Prism 10 software was used to perform statistical analysis and hypothesis testing. All resulting values are reported as mean and standard error of the mean. Spheroid-based experiments were conducted with 3-6 biological replicates containing 10-25 technical replicates (spheroids) each. Drug response IC₅₀ values were generated by normalizing MTS-mediated absorbance values to untreated controls. Similarly, fold changes in spheroid proliferation and migration were calculated by normalizing absorbance values to a day 0 control for each condition. All qPCR data was compared to control conditions within the experiment and performed in triplicate.

Other objects, features and advantages of the disclosure will become apparent from the foregoing figures, detailed description, and examples. It should be understood, however, that the figures, detailed description, and examples, while indicating specific embodiments of the disclosure, are given by way of illustration only and are not meant to be limiting. Additionally, it is contemplated that changes and modifications within the spirit and scope of the disclosure will become apparent to those skilled in the art from the detailed description. In further embodiments, features from specific embodiments may be combined with features from other embodiments. For example, features from one embodiment may be combined with features from any of the other embodiments. In further embodiments, additional features may be added to the specific embodiments described herein.

What is claimed is:

1. A nanoparticle composition for treatment of ovarian cancer comprising a small interfering RNA targeting expression of a signal regulatory protein-a gene (SIRPα siRNA).

2. The nanoparticle composition of claim 1, wherein the nanoparticle is a lipid-based nanoparticle.

3. The nanoparticle composition of claim 1, wherein the nanoparticle contains an ionizable lipid with a preference for localization in a liver.

4. The nanoparticle composition of claim 1, wherein the SIRPα siRNA includes sense and antisense sequences of SEQ ID NO. 1 and SEQ ID NO. 2.

5. The nanoparticle composition of claim 1, wherein the SIRPα siRNA includes sense and antisense sequences of SEQ ID NO. 3 and SEQ ID NO. 4.

6. A pharmaceutical composition comprising a therapeutically effective amount of the nanoparticle composition of claim 1.

7. A method for treating a metastatic tissue, comprising:
contacting the metastatic tissue with the nanoparticle composition of claim 1.

8. The method of claim 7, wherein the tissue is a metastatic ovarian cancer.

9. The method of claim 8, wherein the metastatic ovarian cancer presents in the liver.

10. The method of claim 7, further comprising:
contacting the tissue with a platinum-based chemotherapeutic agent.

11. The method of claim 10, wherein the platinum-based chemotherapeutic agent is one or more of cisplatin, carboplatin, oxaliplatin, and picoplatin.

12. The method of claim 10, wherein the metastatic tissue is concurrently contacted with the nanoparticle composition of claim 1 and the platinum-based chemotherapeutic agent.

13. A method of treating a subject diagnosed as having ovarian cancer, the method comprising: administering to the subject a therapeutically effective amount of the nanoparticle composition of claim 1.

14. The method of claim 13, wherein the nanoparticle is a lipid-based nanoparticle.

15. The method of claim 14, wherein the nanoparticle contains an ionizable lipid or a cationic lipid with a preference for localization in a liver.

16. The method of claim 13, wherein the SIRPα siRNA include sense and antisense sequences of SEQ ID NO. 1 and SEQ ID NO. 2.

17. The method of claim 13, wherein the SIRPα siRNA include sense and antisense sequences of SEQ ID NO. 3 and SEQ ID NO. 4.

18. The method of claim 13, wherein the ovarian cancer is concomitant with metastasis.

19. The method of claim 13, further comprising:
administering to the subject a therapeutically effective amount of a platinum-based chemotherapeutic agent.

20. The method of claim 19, wherein the platinum-based chemotherapeutic agent is one or more of cisplatin, carboplatin, oxaliplatin, and picoplatin.
